(19) United States
(12) Patent Application Publication
(10) Pub. No.: US 2026/0284630 A1
(43) Pub. Date: Sep. 24, 2026

(54) DIGITAL MICROFLUIDIC CHIP, AND METHOD FOR PERFORMING DROPLET GENERATION AND LIBRARY PREPARATION USING SAME

(71) Applicants: BEIJING BOE SENSOR TECHNOLOGY CO., LTD., Beijing (CN); BOE TECHNOLOGY GROUP CO., LTD., Beijing (CN)

(72) Inventors: Bolin FAN, Beijing (CN); Le GU, Beijing (CN); Yongjia GAO, Beijing (CN); Li YANG, Beijing (CN); Huazhe LIU, Beijing (CN)

(21) Appl. No.: 18/996,091

(22) PCT Filed: Apr. 28, 2023
(86) PCT No.: PCT/CN2023/091673

§ 371 Date: Jan. 17, 2025

(51) Int. Cl. B01J 19/00; C12N 15/10

(52) U.S. Cl. CPC B01J 19/0046; C12N 15/1093; B01J 2219/00479; B01J 2219/00722

(57) ABSTRACT

A digital microfluidic chip and methods for droplet generation and library preparation using the same, the digital microfluidic chip includes: a droplet generation structure, configured to generate droplets of various volumes, and the droplet generation structure includes: one or more droplet generation modules, wherein each droplet generation module is configured to generate at least one of a first droplet, a second droplet, and a third droplet, and the first droplet, the second droplet, and the third droplet respectively have a first volume, a second volume, and a third volume that are different from each other; a droplet receiving module, configured to achieve at least one of mixing and reaction of a plurality of droplets; and an interconnection module, configured to drive the first droplet, the second droplet, and the third droplet, and to connect the droplet generation structure to the droplet receiving module.

### TECHNICAL FIELD

Embodiments of the present disclosure relate to a digital microfluidic chip and methods for droplet generation and library preparation using the same.

### BACKGROUND

Microfluidic technology is a technology that can accurately control and manipulate micro-scale fluids, capable of integrating basic operations such as sample preparation, reaction, separation, and detection in biochemical processes into a centimeter-level chip. Microfluidic technology is generally applied in the analysis process of micro-doses of drugs in fields such as biology, chemistry, and pharmaceuticals. Digital microfluidic chips treat a droplet as the object of manipulation and control the movement of the droplet through forces such as electric field force, thermal force, electrostatic force, etc., with the ability to control each individual droplet separately. Microfluidic technology requires only small amount of sample and reagent, has a simple structure, and facilitates large-scale integration. The manipulation of droplets is more convenient; droplet size and shape are controllable, offering advantages such as miniaturization, integration, low cost, high sensitivity, and high flexibility. This significantly speeds up the processes of moving, extracting, separating, mixing, and detecting samples, showing great potential in many fields such as physics, chemistry, biology, and medicine, etc.

Digital microfluidic chips are particularly suitable for complex biochemical reaction processes. Due to their cost-effectiveness, passive digital microfluidic chips have become the mainstream chip solution in commercially available digital microfluidic products. However, passive digital microfluidic chips face issues such as a limited number of controllable electrodes and low flexibility in the volume of liquid manipulation. In biochemical processes, multiple volume types of droplets may be required, and the range of volumes of droplets may be quite large. Therefore, how to improve the range and flexibility of volumes of droplet manipulation in digital microfluidic chips is a technical challenge currently being faced.

### SUMMARY

At least one embodiment of the present disclosure provides a digital microfluidic chip, including: a droplet generation structure, configured to generate droplets of various volumes, and the droplet generation structure includes: one or more droplet generation modules, wherein each droplet generation module is configured to generate at least one of a first droplet, a second droplet, and a third droplet, and the first droplet, the second droplet, and the third droplet respectively have a first volume, a second volume, and a third volume that are different from each other; a droplet receiving module, configured to achieve at least one of mixing and reaction of a plurality of droplets; and an interconnection module, configured to drive the first droplet, the second droplet, and the third droplet, and to connect the droplet generation structure to the droplet receiving module.

In the digital microfluidic chip provided by at least one embodiment of the present disclosure, the interconnection module includes: one or more droplet driving modules, wherein each droplet driving module is configured to drive at least one of the first droplet, the second droplet, and the third droplet, and a volume of a drivable droplet of a single driving electrode in the one or more droplet driving module is the first volume, the second volume, or the third volume.

In the digital microfluidic chip provided by at least one embodiment of the present disclosure, the one or more droplet driving modules include: at least one first droplet driving module, wherein a single driving electrode in each first droplet driving module is configured to drive the first droplet; at least one second droplet driving module, wherein a single driving electrode in each second droplet driving module is configured to drive the second droplet; and at least one third droplet driving module, wherein a single driving electrode in each third droplet driving module is configured to drive the third droplet.

In the digital microfluidic chip provided by at least one embodiment of the present disclosure, the second volume is larger than a minimum volume of a drivable droplet of the first droplet driving module, and is smaller than a maximum volume of the drivable droplet of the first droplet driving module; and the third volume is larger than a minimum volume of a drivable droplet of the second droplet driving module, and is smaller than a maximum volume of the drivable droplet of the second droplet driving module.

In the digital microfluidic chip provided by at least one embodiment of the present disclosure, the first volume is smaller than the second volume and the third volume, both the second volume and the third volume are integer multiples of the first volume, and the second volume and the third volume are different from each other.

In the digital microfluidic chip provided by at least one embodiment of the present disclosure, the second volume is twice the first volume, and the third volume is five times the first volume; the first volume is a minimum unit volume of a droplet that is manipulatable by the digital microfluidic chip, and the digital microfluidic chip is configured to manipulate liquid with a volume range from the first volume to twenty times the first volume and a step size of the minimum unit volume.

In the digital microfluidic chip provided by at least one embodiment of the present disclosure, the one or more droplet generation modules include: at least one first droplet generation module, wherein each first droplet generation module is configured to generate the first droplet; at least one second droplet generation module, wherein each second droplet generation module is configured to generate the second droplet; and at least one third droplet generation module, wherein each third droplet generation module is configured to generate the third droplet.

In the digital microfluidic chip provided by at least one embodiment of the present disclosure, wherein each of the first droplet generation module, the second droplet generation module, and the third droplet generation module includes a first driving electrode, a second driving electrode, a third driving electrode, a fourth driving electrode, a fifth driving electrode, and a sixth driving electrode; and a volume of a droplet that can be accommodated and driven by a driving electrode with a smallest area in each droplet generation module is approximately equal to a volume of a single droplet generated by the each droplet generation module.

In the digital microfluidic chip provided by at least one embodiment of the present disclosure, in each of the first droplet generation module, the second droplet generation module, and the third droplet generation module, the second driving electrode, the third driving electrode, the fourth driving electrode, and the sixth driving electrode each have a first area and are arranged sequentially in a first sub-direction, and the sixth driving electrode is connected to a driving electrode in the interconnection module; the fifth driving electrode includes two fifth driving sub-electrodes that are electrically connected to each other, and the two fifth sub-electrodes are arranged on opposite sides of the second driving electrode and the third driving electrode in a second sub-direction perpendicular to the first sub-direction, and each of the two fifth driving sub-electrodes has a second area; and the first driving electrode is disposed on a side of the second driving electrode away from the third driving electrode in the first sub-direction, and is connected to the second driving electrode and the fifth driving electrode, and the first driving electrode has a third area, wherein the first area is smaller than the second area, and the second area is smaller than the third area.

In the digital microfluidic chip provided by at least one embodiment of the present disclosure, each of the first droplet generation module and the second droplet generation module further includes an additional driving electrode, the additional driving electrode is located on a side of the first driving electrode away from the second driving electrode and the fifth driving electrode, and the additional driving electrode has the third area same as that of a corresponding first driving electrode.

In the digital microfluidic chip provided by at least one embodiment of the present disclosure, each of the first droplet generation module and the second droplet generation module further includes an additional dummy electrode, and the additional dummy electrode is located on a side of the additional driving electrode away from the first driving electrode.

In the digital microfluidic chip provided by at least one embodiment of the present disclosure, the first area, the second area, and the third area of corresponding driving electrodes in the first droplet generation module are smaller than the first area, the second area, and the third area of corresponding driving electrodes in the second droplet generation module, respectively; and the first area, the second area, and the third area of the corresponding driving electrodes in the second droplet generation module are smaller than the first area, the second area, and the third area of corresponding driving electrodes in the third droplet generation module, respectively.

In the digital microfluidic chip provided by at least one embodiment of the present disclosure, the one or more droplet generation modules include: at least one fourth droplet generation module, wherein each fourth droplet generation module is configured to generate at least two of the first droplet, the second droplet, and the third droplet.

In the digital microfluidic chip provided by at least one embodiment of the present disclosure, wherein each fourth droplet generation module includes a first driving electrode, a second driving electrode, a third driving electrode, a fourth driving electrode, a fifth driving electrode, a sixth driving electrode, a first additional driving electrode, a second additional driving electrode, and two third additional driving electrodes; the second driving electrode, the third driving electrode, the fourth driving electrode, the sixth driving electrode, the first additional driving electrode, and the second additional driving electrode are arranged sequentially in a first sub-direction, and the two third additional driving electrodes are arranged on opposite sides of the sixth driving electrode in a second sub-direction perpendicular to the first sub-direction; the second driving electrode, the third driving electrode, and the fourth driving electrode each have a first body area, and a sum of areas of the sixth driving electrode and the two third additional driving electrodes is approximately equal to the first body area, and the first additional driving electrode and the second additional driving electrode each have a second body area; the fifth driving electrode includes two fifth sub-electrodes, which are located on opposite sides of the second driving electrode and the third driving electrode in the second sub-direction; and the first driving electrode is disposed on a side of the second driving electrode and the fifth driving electrode away from the fourth driving electrode.

In the digital microfluidic chip provided by at least one embodiment of the present disclosure, the fourth droplet generation module is configured to generate the second droplet, the third droplet, or an additional droplet, a volume of the additional droplet is approximately equal to the third volume minus the second volume, and a volume of a droplet that can be accommodated and driven by each driving electrode having the second body area is approximately equal to the second volume of the second droplet, and a volume of a droplet that can be accommodated and driven by a single driving electrode or a combination of driving electrodes having the first body area is approximately equal to the third volume of the third droplet; or the fourth droplet generation module is configured to generate the first droplet or the second droplet, a volume of a droplet that can be accommodated and driven by each driving electrode having the second body area is approximately equal to the first volume of the first droplet, and a volume of a droplet that can be accommodated and driven by a single driving electrode or a combination of driving electrodes having the first body area is approximately equal to the second volume of the second droplet.

In the digital microfluidic chip provided by at least one embodiment of the present disclosure, the second additional driving electrode is connected to a driving electrode in the interconnection module.

In the digital microfluidic chip provided by at least one embodiment of the present disclosure, further including a fourth additional driving electrode and a fifth additional driving electrode, disposed on a side of the second additional driving electrode away from the first additional driving electrode in the first sub-direction; wherein the fourth additional driving electrode and the fifth additional driving electrode each have a third body area, and the third body area is smaller than the second body area.

In the digital microfluidic chip provided by at least one embodiment of the present disclosure, the droplet receiving module is configured to at least implement mixing and a first reaction of liquid within a first mixing volume range, and implement mixing and a second reaction of liquid within a second mixing volume range.

In the digital microfluidic chip provided by at least one embodiment of the present disclosure, the droplet receiving module includes: a first reaction region, configured to at least implement the mixing and the first reaction of the liquid within the first mixing volume range, wherein the first reaction region is connected to a third droplet driving module of the interconnection module.

In the digital microfluidic chip provided by at least one embodiment of the present disclosure, the first reaction region includes a plurality of first reaction electrodes arranged in an array, a volume of a droplet that can be accommodated by each first reaction electrode is greater than the first volume and the second volume and less than the third volume, and a maximum value of the first mixing volume range of a mixed droplet that can be accommodated by the plurality of first reaction electrodes is an integer multiple of the third volume.

In the digital microfluidic chip provided by at least one embodiment of the present disclosure, further including: a second reaction region, configured to implement the mixing and the second reaction of the liquid within the second mixing volume range, wherein the second reaction region includes a plurality of second reaction electrodes, and a volume of a droplet that can be accommodated and driven by each second reaction electrode is an integer multiple of the third volume.

In the digital microfluidic chip provided by at least one embodiment of the present disclosure, the second reaction region and the third droplet driving module share a same electrode array.

In the digital microfluidic chip provided by at least one embodiment of the present disclosure, the second reaction region includes a temperature control region, and the temperature control region is configured to control liquid in the temperature control region to react at a temperature within a predetermined temperature range.

In the digital microfluidic chip provided by at least one embodiment of the present disclosure, the temperature control region includes a first temperature control region, a second temperature control region, and a third temperature control region, which are configured to control liquid therein to react at a temperature within a first temperature range, a second temperature range, and a third temperature range that are different from each other, respectively.

In the digital microfluidic chip provided by at least one embodiment of the present disclosure, the droplet generation structure and the interconnection module are located between the first reaction region and the second reaction region.

In the digital microfluidic chip provided by at least one embodiment of the present disclosure, the first reaction region is served as a common reaction region, and is configured to implement the mixing and the first reaction of the liquid within the first mixing volume range, and implement the mixing and the second reaction of the liquid within the second mixing volume range in different operational steps.

In the digital microfluidic chip provided by at least one embodiment of the present disclosure, the droplet generation structure further includes a droplet cutting module, connected to the one or more droplet generation modules, and is configured to cut a droplet from the one or more droplet generation modules into a droplet having a target volume.

In the digital microfluidic chip provided by at least one embodiment of the present disclosure, the droplet cutting module and a first droplet driving module in the interconnection module share a same electrode array which is directly connected to the first droplet generation module.

In the digital microfluidic chip provided by at least one embodiment of the present disclosure, the droplet cutting module includes a plurality of main cutting electrodes, and a volume of a droplet that can be accommodated and driven by each main cutting electrode is approximately equal to the first volume.

In the digital microfluidic chip provided by at least one embodiment of the present disclosure, the plurality of main cutting electrodes are arranged along a first direction, and an end main cutting electrode, at an end of the droplet cutting module in the first direction, of the plurality of main cutting electrodes is connected to the interconnection module.

In the digital microfluidic chip provided by at least one embodiment of the present disclosure, one or more first droplet generation modules for generating the first droplet, among the one or more droplet generation modules, are located on one side or opposite two sides of the droplet cutting module in a second direction perpendicular to the first direction.

In the digital microfluidic chip provided by at least one embodiment of the present disclosure, the droplet cutting module further includes an auxiliary cutting electrode, the auxiliary cutting electrode is connected to a main cutting electrode close to the interconnection module, and also connected to the interconnection module.

In the digital microfluidic chip provided by at least one embodiment of the present disclosure, the plurality of main cutting electrodes each have a first width, the auxiliary cutting electrode has a second width, and the second width is greater than the first width, the first width and the second width are widths along a first direction.

In the digital microfluidic chip provided by at least one embodiment of the present disclosure, the auxiliary cutting electrode is located on opposite two sides of the main cutting electrode close to the interconnection module, in a second direction perpendicular to the first direction.

In the digital microfluidic chip provided by at least one embodiment of the present disclosure, a number of the at least one second droplet driving module is greater than a number of the at least one first droplet driving module and a number of the at least one third droplet driving module.

In the digital microfluidic chip provided by at least one embodiment of the present disclosure, a droplet generation module for generating the second droplet and the third droplet, among the one or more droplet generation modules, is directly connected to one or more second droplet driving modules.

In the digital microfluidic chip provided by at least one embodiment of the present disclosure, including: a first substrate, including: a first base substrate; a first conductive layer, located on a side of the first base substrate; a second conductive layer, located on a side of the first conductive layer away from the base substrate, wherein corresponding portions of the first conductive layer and the second conductive layer are electrically connected to each other; a dummy layer, located between the first conductive layer and the second conductive layer, and being electrically floating or grounded; and a first hydrophobic layer, located on a side of the second conductive layer away from the first base substrate.

In the digital microfluidic chip provided by at least one embodiment of the present disclosure, the second conductive layer includes a plurality of driving electrodes of the droplet generation structure, the droplet receiving module, and the interconnection module, and the plurality of driving electrodes are spaced apart from each other; and the dummy layer includes a plurality of dummy electrodes spaced apart from each other, and an outer contour of an orthogonal projection of each dummy electrode on the first base substrate coincides with an outer contour of an orthographic projection of a corresponding driving electrode among the plurality of driving electrodes on the base substrate.

In the digital microfluidic chip provided by at least one embodiment of the present disclosure, the plurality of dummy electrodes each have an opening, and the plurality of driving electrodes are respectively connected to the first conductive layer through a plurality of conductive vias, and the plurality of conductive vias are respectively located in corresponding openings of the plurality of dummy electrodes and spaced apart from the plurality of dummy electrodes.

In the digital microfluidic chip provided by at least one embodiment of the present disclosure, the first conductive layer further includes a plurality of conductive wires, and the plurality of driving electrodes are connected to bonding electrodes through the plurality of conductive wires.

In the digital microfluidic chip provided by at least one embodiment of the present disclosure, the first conductive layer and the second conductive layer include a transparent conductive material, and the dummy layer includes an opaque conductive material.

In the digital microfluidic chip provided by at least one embodiment of the present disclosure, the dummy layer includes a metal material.

In the digital microfluidic chip provided by at least one embodiment of the present disclosure, the dummy layer further includes an additional dummy electrode of the one or more droplet generation modules, and an orthogonal projection of the additional dummy electrode on the first base substrate do not overlap with the second conductive layer.

In the digital microfluidic chip provided by at least one embodiment of the present disclosure, further including: a second substrate, arranged opposite to the first substrate, wherein the second substrate includes: a second base substrate; a third conductive layer, located on a side of the second base substrate; and a second hydrophobic layer, located on a side of the third conductive layer away from the second base substrate; and a droplet channel, located between the first hydrophobic layer of the first substrate and the second hydrophobic layer of the second substrate, wherein the second substrate is provided with a plurality of sample inlets penetrating through the second substrate, and the plurality of sample inlets are respectively arranged corresponding to the plurality of droplet generation modules; and for a droplet generation module including the additional dummy electrode, an orthogonal projection of the additional dummy electrode on a main surface of the first substrate facing the second substrate overlaps with an orthogonal projection of a corresponding sample inlet on the main surface of the first substrate.

In the digital microfluidic chip provided by at least one embodiment of the present disclosure, the one or more droplet generation modules include a droplet generation module without an additional dummy electrode, and an orthogonal projection of a sample inlet corresponding to the droplet generation module on the main surface of the first substrate is close to an orthogonal projection of a driving electrode of the droplet generation module on the main surface of the first substrate, and does not overlap with the orthogonal projection of the driving electrode.

At least one embodiment of the present disclosure provides a method for generating droplets of various volumes using the droplet generation structure in any one of the above-mentioned digital microfluidic chips, including at least one of the following: generating one or more first droplets each having the first volume using the droplet generation module; generating one or more second droplets each having the second volume using the droplet generation module; generating one or more third droplets each having the third volume using the droplet generation module, wherein the first volume is smaller than the second volume, and the second volume is smaller than the third volume; generating at least one of one or more fourth droplets each having a fourth volume and one or more fifth droplets each having a fifth volume using the droplet generation module and a droplet cutting module in combination, wherein the fourth volume and the fifth volume are greater than the second volume and smaller than the third volume; and mixing at least two of the one or more first droplets, the one or more second droplets, the one or more third droplets, the one or more fourth droplets, and the one or more fifth droplets to obtain droplets with other target volumes.

In the above method provided by at least one embodiment of the present disclosure, generating the fourth droplet using the droplet generation module and the droplet cutting module in combination, includes: generating an initial droplet with the third volume using a third droplet generation module; and cutting out two cut droplets each having the first volume from the initial droplet by the droplet cutting module, and moving the two cut droplets to a waste liquid region, wherein a remaining droplet of the initial droplet after being cut forms the fourth droplet.

In the above method provided by at least one embodiment of the present disclosure, generating the fifth droplet using the droplet generation module and the droplet cutting module in combination, includes: generating an initial droplet with the third volume using a third droplet generation module; and cutting out a cut droplet having the first volume from the initial droplet by the droplet cutting module, and moving the cut droplet to a waste liquid region, wherein a remaining droplet of the initial droplet after being cut forms the fifth droplet.

At least one embodiment of the present disclosure provides a method for preparing a gene library using any one of the above-mentioned digital microfluidic chips, including: adding a deoxyribonucleotide sample solution, a ligase buffer solution, a linker solution, and a ligase solution to corresponding droplet generation modules of the one or more droplet generation modules, respectively, and generating a sample droplet, a ligase buffer droplet, a linker droplet, and a ligase droplet through the droplet generation structure; moving the sample droplet, the ligase buffer droplet, the linker droplet, and the ligase droplet to the droplet receiving module through the interconnection module, wherein the sample droplet, the ligase buffer droplet, the linker droplet, and the ligase droplet are mixed and reacted in a first reaction region of the droplet receiving module to form a sample mixture droplet; performing deoxyribonucleotide adsorption and elution on the sample mixture droplet to obtain a first deoxyribonucleotide chain; adding an amplification solution and a primer solution to corresponding droplet generation modules among the one or more droplet generation modules, respectively, and generating an amplification droplet and a primer droplet through the droplet generation structure; moving the amplification droplet and the primer droplet to the droplet receiving module through the interconnection module, wherein the amplification droplet and the primer droplet with the first deoxyribonucleotide chain undergo a polymerase chain reaction in the droplet receiving module to form an amplified deoxyribonucleotide mixture droplet; and performing deoxyribonucleotide adsorption and elution on the amplified deoxyribonucleotide mixture droplet to obtain an amplified deoxyribonucleotide chain.

In the above method provided by at least one embodiment of the present disclosure, the sample droplet has a sixth volume, and generating the sample droplet includes: adding the deoxyribonucleotide sample solution to a third droplet generation module; generating a first sample droplet and a second sample droplet each having the third volume through the third droplet generation module; moving the second sample droplet to a droplet cutting module through the interconnection module; and cutting the second sample droplet into a cut sample droplet having the second volume and a third sample droplet having a remaining volume by the droplet cutting module, wherein the first sample droplet and the third sample droplet constitute the sample droplet having the sixth volume.

In the above method provided by at least one embodiment of the present disclosure, generating the ligase buffer droplet, the linker droplet, and the ligase droplet includes: adding the ligase buffer solution to a second droplet generation module, and generating the ligase buffer droplet having the second volume through the second droplet generation module; adding the ligase solution to the second droplet generation module, and generating two ligase sub-droplets each having the second volume through the second droplet generation module, wherein the two ligase sub-droplets constitute the ligase droplet; and adding the linker solution to a first droplet generation module, and generating the linker droplet having the first volume through the first droplet generation module.

### BRIEF DESCRIPTION OF DRAWINGS

In order to clearly illustrate the technical solution of the embodiments of the present disclosure, the drawings of the embodiments will be briefly described. It is obvious that the described drawings in the following are only related to some embodiments of the present disclosure and thus are not limitative of the present disclosure.

FIG. 1A is a schematic top view of a digital microfluidic chip according to some embodiments of the present disclosure; FIG. 1B is a schematic top view of an upper substrate in a digital microfluidic chip according to some embodiments of the present disclosure; FIG. 1C is a schematic cross-sectional view taken along line I-I′ in FIG. 1A of a digital microfluidic chip according to some embodiments of the present disclosure;

FIG. 2A to FIG. 2C illustrate top views of a driving electrode structure in a digital microfluidic chip according to some embodiments of the present disclosure;

FIG. 3A to FIG. 3C are schematic top views of various droplet generation modules in a digital microfluidic chip according to some embodiments of the present disclosure;

FIG. 4A to FIG. 4C are schematic top views of various droplet generation modules in a digital microfluidic chip according to some embodiments of the present disclosure;

FIG. 5A to FIG. 5K are schematic top views of driving methods for generating various volumes of droplets using droplet generation modules according to some embodiments of the present disclosure;

FIG. 6 is a schematic top view of a partial region of a droplet receiving module according to some embodiments of the present disclosure;

FIG. 7A to FIG. 7D are schematic top views of various electrode arrays in a digital microfluidic chip according to some embodiments of the present disclosure;

FIG. 8A to FIG. 8C illustrate schematic diagrams for generating a droplet having a target volume using a digital microfluidic chip through a droplet generation module and a droplet cutting module;

FIG. 9A illustrates a top view of respective modules in a digital microfluidic chip according to some other embodiments of the present disclosure; FIG. 9B illustrates an enlarged plan view of a droplet cutting module in the digital microfluidic chip of FIG. 9A;

FIG. 10A and FIG. 10B are schematic diagrams of cutting droplets using the droplet cutting module shown in FIG. 9A and FIG. 9B according to some embodiments of the present disclosure;

FIG. 11A and FIG. 11B are schematic top views of respective modules in a digital microfluidic chip according to some other embodiments of the present disclosure; and

FIG. 12A to FIG. 12I illustrate schematic top views of a method for preparing a gene library using a digital microfluidic chip according to some embodiments of the present disclosure.

### DETAILED DESCRIPTION

In order to make objects, technical details and advantages of the embodiments of the present disclosure apparent, the technical solutions of the embodiments will be described in a clearly and fully understandable way in connection with the drawings related to the embodiments of the present disclosure. Apparently, the described embodiments are just a part but not all of the embodiments of the present disclosure. Based on the described embodiments herein, those skilled in the art can obtain other embodiment(s), without any inventive work, which should be within the scope of the disclosure.

Unless otherwise defined, all the technical and scientific terms used herein have the same meanings as commonly understood by one of ordinary skill in the art to which the present disclosure belongs. The terms “first,” “second,” etc., used in the present disclosure are not intended to indicate any sequence, amount or importance, but distinguish different components. The terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not limited to a physical connection or mechanical connection, but may also include an electrical connection, directly or indirectly.

Various embodiments of the present disclosure provide a digital microfluidic chip that can solve the technical problems of existing digital microfluidic chips such as low flexibility in liquid manipulation volume, few types and small range of manipulable droplet volumes. The digital microfluidic chip provided by the embodiments of the present disclosure is capable of generating droplets of multiple volume types and can achieve manipulation of droplets of multiple volume types and with a wide range of volumes, and the droplet manipulation is highly flexible and can be applied to more biochemical reaction processes.

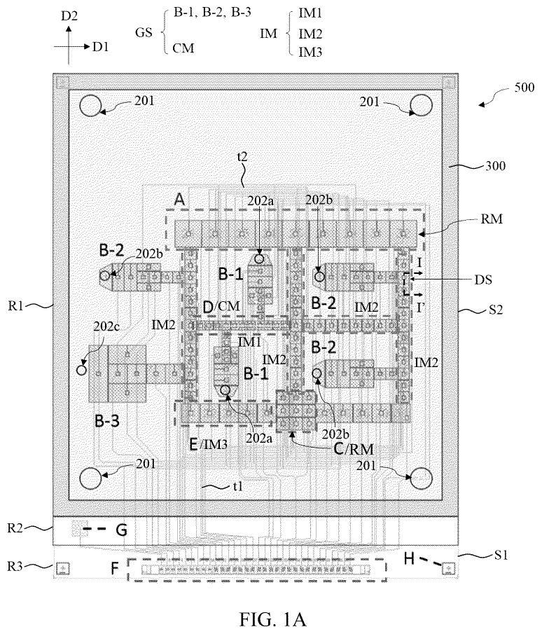

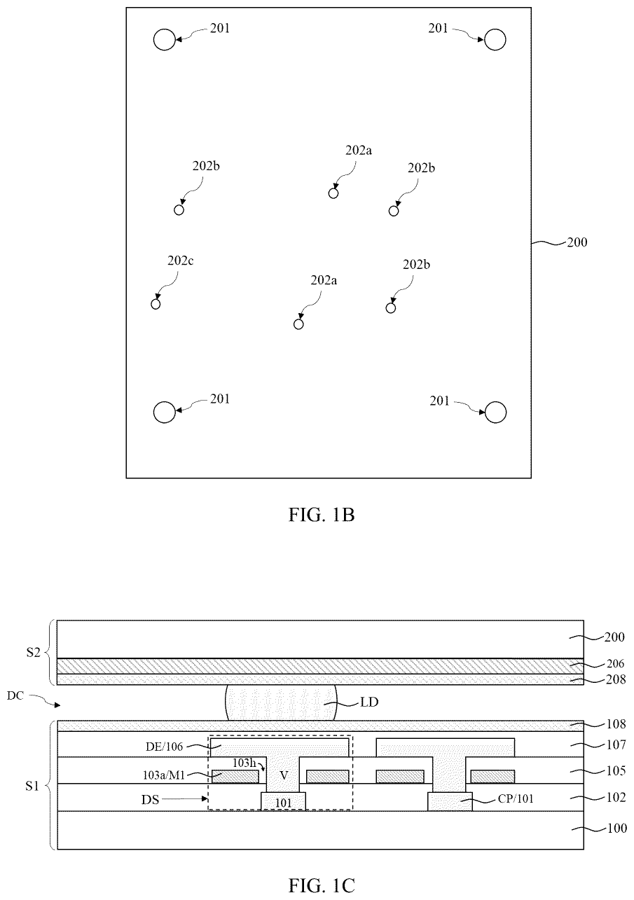

FIG. 1A is a schematic top view of a digital microfluidic chip according to some embodiments of the present disclosure; FIG. 1B is a schematic top view of an upper substrate in a digital microfluidic chip according to some embodiments of the present disclosure; and FIG. 1C is a schematic cross-sectional view taken along line I-I′ in FIG. 1A of a digital microfluidic chip according to some embodiments of the present disclosure.

Referring to FIG. 1, in some embodiments, a digital microfluidic chip 500 includes a droplet generation structure GS, a droplet receiving module RM, and an interconnection module IM. The droplet generation structure GS is configured to generate droplets of various volumes and may include one or more droplet generation modules. For example, each droplet generation module is configured to generate at least one of a first droplet, a second droplet, and a third droplet, and the first droplet, the second droplet, and the third droplet respectively have a first volume, a second volume, and a third volume that are different from each other. The droplet receiving module RM is configured to achieve at least one of mixing and reaction of a plurality of droplets. The interconnection module IM is configured to drive the first droplet, the second droplet, the third droplet, and other droplets of various volumes, and to connect respective modules and/or regions, such as connect the respective modules in the droplet generation structure GS to each other, and connect the droplet generation structure GS to the droplet receiving module RM and/or to different regions in the droplet receiving module RM, thereby implementing operations such as driving droplets to move between the respective modules and/or regions.

In the microfluidic chip, the more volume types of a single droplet that can be generated by the droplet generation module, the easier and more efficient the droplet manipulation will be. In addition, in order to improve the utilization efficiency of the chip area, it is necessary to provide as few droplet generation modules as possible. In the embodiments of the present disclosure, taking into account both of the above factors, the initial single droplet volume types that can be generated by a plurality of droplet generation modules are set to three types, namely the first volume, the second volume, and the third volume, and through the cooperative use of respective modules, the flexibility of droplet volume manipulation of the microfluidic chip can be enhanced, thereby achieving the effect that the volumes of manipulatable droplets are of a large variety types and in a wide span, and enabling droplet manipulation simple, with high operational efficiency and high chip area utilization. Here, the initial single droplet volume refers to an initial volume of a single droplet generated by the droplet generation module, that is, the volume of a single droplet that has not been cut and/or mixed after being generated by the droplet generation module.

In some embodiments, the interconnection module IM may include one or more droplet driving modules, each droplet driving module is configured to drive at least one of the first droplet, the second droplet, and the third droplet, and the volume of a drivable droplet of a single driving electrode in the droplet driving module is the first volume, the second volume, or the third volume. For example, the first volume is smaller than the second volume and the third volume, and the droplet driving module for driving the first droplet and the droplet generation module for generating the first droplet may be directly connected.

In some embodiments, the one or more droplet driving modules may include at least one droplet driving module IM1, at least one droplet driving module IM2, and at least one droplet driving module IM3. The droplet driving modules IM1, IM2, and IM3 may be referred to as a first droplet driving module, a second droplet driving module and a third droplet driving module, respectively. For example, a single driving electrode in each first droplet driving module IM1 is configured to drive the first droplet, a single driving electrode in each second droplet driving module IM2 is configured to drive the second droplet, and a single driving electrode in each third droplet driving module IM3 is configured to drive the third droplet. For example, the volume of a drivable droplet of each driving electrode in the first droplet driving module IM1 is the first volume, the volume of a drivable droplet of each driving electrode in the second droplet driving module IM2 is the second volume, and the volume of a drivable droplet of each driving electrode in the third droplet driving module IM3 is the third volume.

In some embodiments, the volume of the drivable droplet of a single driving electrode in each driving module is the minimum volume of a drivable droplet that can be driven by the driving module; that is, the minimum volumes of the drivable droplets of the droplet driving modules IM1, IM2, and IM3 are the first volume, the second volume, and the third volume, respectively. It should be understood that, each droplet driving module may include a plurality of driving electrodes, and may drive droplet(s) through the cooperative operation of one or more driving electrodes. That is, in addition to being configured to drive the first, second, and third droplets, the droplet driving modules IM1, IM2, and IM3 may also drive droplets of other volumes through corresponding plurality of driving electrodes. For example, considering the accuracy of droplet manipulation, the volume of the droplet that can be accommodated and driven by three driving electrodes in each droplet driving module is defined as the maximum volume of the drivable droplet that can be driven by the droplet driving module; that is, the maximum volumes of the drivable droplets of the droplet driving modules IM1, IM2, and IM3 are three times the first volume, three times the second volume, and three times the third volume, respectively. Therefore, the volume range of the drivable droplet of the droplet driving module IM1 may be from the first volume to three times the first volume; the volume range of the drivable droplet of the droplet driving module IM2 may be from the second volume to three times the second volume; and the volume range of the drivable droplet of the droplet driving module IM3 may be from the third volume to three times the third volume.

In some embodiments, the second volume is larger than the minimum volume of the drivable droplet of the first droplet driving module, and is smaller than the maximum volume of the drivable droplet of the first droplet driving module; and the third volume is larger than the minimum volume of the drivable droplet of the second droplet driving module, and is smaller than the maximum volume of the drivable droplet of the second droplet driving module.

In some embodiments, the first volume is smaller than the second volume and the third volume; for example, the first volume, the second volume, and the third volume are sequentially increased. The first volume may be the smallest operation unit of the microfluidic chip, i.e., the smallest volume of a droplet that can be manipulated by microfluidic chip. In some embodiments, the region D directly connected to the droplet generation module (e.g., droplet generation module B-1) that generates the first volume of droplet may be or include the first droplet driving module IM1.

In some embodiments, the droplet generation structure GS further includes one or more droplet cutting modules CM, which is connected to the one or more droplet generation modules, and configured to cut a droplet from the one or more droplet generation modules into a droplet having a target volume. For example, the droplet cutting module CM may be connected to the one or more droplet generation modules through the interconnection module IM1, or the droplet cutting module CM may also be directly connected to the one or more droplet generation modules.

In some embodiments, the droplet cutting module CM and the first droplet driving module IM1 in the interconnection module may share the same electrode array; for example, the region D directly connected to the droplet generation module (e.g., droplet generation module B-1) that generates the first volume of droplet may be the first droplet driving module IM1, and may be multi-used as the droplet cutting module CM, that is, the droplet generation module used to generate the first droplet may be directly connected to the droplet cutting module CM. The droplet cutting module CM includes a plurality of cutting driving electrodes, and the volume of a drivable droplet that can be driven by each cutting driving electrode may be approximately equal to the first volume, but the present disclosure is not limited thereto. In some other embodiments, the droplet cutting module CM may further include a single cutting driving electrode capable of driving a droplet whose volume is larger than the first volume.

In some embodiments, both the second volume and the third volume are integer multiples of the first volume, and the second volume and the third volume are different from each other; for example, the second volume is twice the first volume, and the third volume is five times the first volume. In some embodiments, the first volume is the minimum unit volume of the manipulable droplet that can be manipulated by the digital microfluidic chip, and the digital microfluidic chip can manipulate liquid with the volume range from the first volume to tens of times (for example, twenty times) the first volume and the step size thereof being the minimum unit volume. In the present disclosure, the “step size” of a liquid refers to the volume difference between adjacent manipulatable droplets that can be manipulated.

For example, the droplet generation module may include a droplet generation module configured to generate droplets with the minimum operational unit volumes of 1 μL, 2 μL, and 5 μL, and through the cooperative operation of the droplet generation module and the droplet cutting module, at least droplets of multiple volume types ranging from 1 μL to 20 μL with the step size of 1 μL may be generated, that is, droplets with volumes of 1 μL, 2 μL, 3 μL, 4 μL, 5 μL, 6 μL, 7 μL, 8 μL, 9 μL, 10 μL, 11 μL, 12 μL, 13 μL, 14 μL, 15 μL, 16 μL, 17 μL, 18 μL, 19 μL, 20 μL may be generated.

In some embodiments, the digital microfluidic chip 500 may at least include a sample inlet region, a liquid reaction region, a liquid cutting region, a waste liquid region, and a sampling region. The digital microfluidic chip 500 is, for example, a passive digital microfluidic chip. The sample inlet region is the region where the sample solution is introduced into the digital microfluidic chip. The sample inlet region corresponds to the region where the droplet generation module is located, and the droplet generation module may also be called the sample inlet module. The liquid reaction region may include at least part of the droplet receiving module, for example, may include a region C and a region A shown in FIG. 1A, and regions C and A may also be referred to as reaction regions C and A. The liquid cutting region may at least include the droplet cutting module CM; in some embodiments, each droplet generation module can also achieve droplet cutting. The waste liquid region is used to receive waste droplets, such as waste droplets cut off by the droplet cutting module. The sampling region is a region where target substances (for example, target liquids, products, etc.) are drawn from the digital microfluidic chip. The target substances, for example, may be waste liquid or the product formed by the sample solution after undergoing a series of biochemical reaction processes within the digital microfluidic chip. In some embodiments, the waste liquid region and the sampling region may be selected from the sample inlet modules according to the needs, but other modules except the above modules may also be set separately as the waste liquid region and the sampling region.

In some embodiments, the digital microfluidic chip 500 includes a body region R1, a peripheral region R2 and a bonding region R3. The peripheral region R2 is, for example, on a side of the body region R1 in a direction D2, and the bonding region R3 is on a side of the peripheral region R2 away from the body region R1. The droplet generation structure GS, the droplet receiving module RM, the interconnection module IM and other modules are provided in the body region R1. A plurality of conductive wires (for example, the conductive wires t1 and t2 shown in the figure) extend from respective modules in the body region R1 through the peripheral region R2 to the bonding region R3, so as to electrically connect the driving electrodes in respective modules to corresponding bonding electrodes in a bonding electrode region F of the bonding region R3. The bonding electrodes are electrically connected to a driving integrated circuit (IC), and the driving IC is configured to apply the corresponding driving signals to the driving electrodes in the respective modules through the bonding electrodes and a plurality of conductive wires.

Referring to FIG. 1A to FIG. 1C, in some embodiments, the droplet generation structure GS, the droplet receiving module RM, the interconnection module IM and other modules in the digital microfluidic chip 500 all include similar stacked structures. For example, the digital microfluidic chip 500 includes a first substrate S1, a second substrate S2, and a droplet channel DC. The first substrate S1 and the second substrate S2 are arranged opposite to and spaced apart from each other, and the droplet channel DC is between the first substrate S1 and the second substrate S2. The first substrate and the second substrate may be referred to as a lower substrate and an upper substrate, respectively. The modules in the microfluidic chip 500 may each be constituted by stacked structures at corresponding positions in the first substrate S1 and the second substrate and corresponding parts of the droplet channel DC. In the process of using the digital microfluidic chip, the droplet LD is moved in the droplet channel DC by applying a driving signal to the driving electrodes in the corresponding module. For example, the digital microfluidic chip may drive the droplet LD to move through the principle of dielectric wetting, and drive the droplet to move in a specific direction by changing the potential between the driving electrode and the droplet LD.

Referring to FIG. 1C, in some embodiments, the first substrate S1 may include a base substrate 100, a conductive layer 101, an insulation layer 102, a dummy layer M1, an insulation layer 105, a conductive layer 106, a dielectric layer 107 and a hydrophobic layer 108. The conductive layer 101 is disposed on a side of the base substrate 100, the conductive layer 106 is disposed on a side of the conductive layer 101 away from the base substrate 100, and corresponding portions of the conductive layer 106 and the conductive layer 101 are electrically connected to each other. The dummy layer M1 is disposed between the conductive layer 101 and the conductive layer 106 and is electrically isolated from the conductive layers 101 and 106. In some embodiments, the dummy layer M1 is electrically floating, that is, not connected to any other conductive component and/or voltage source; alternatively, the dummy layer M1 may also be grounded.

For example, the conductive layer 106 may include a plurality of driving electrodes DE, a part of a plurality of conductive wires (e.g., the horizontally extending conductive wire t2 in FIG. 1A), and/or a part of bonding electrodes; the conductive layer 101 may include conductive connection pads CP, a plurality of conductive wires (e.g., the vertically extending conductive wire t1 in FIG. 1A), and at least a part of the bonding electrodes in the bonding electrode region F; the conductive wires t1 and t2 may be electrically connected to each other through vias (not shown) in the insulation layers 105 and 102; and the conductive connection pad CP may be disposed at an end of the conductive wire t1, and the driving electrode DE is electrically connected to the conductive connection pad CP through the conductive via V, thus connected to the conductive wires t1 and t2, and is further connected to the bonding electrodes through the conductive wires t1 and t2. The dummy layer M1 may include a plurality of dummy electrodes 103a.

Adjacent conductive patterns of the conductive layer 102, the conductive layer 106, and the dummy layer M1 are separated by an insulation layer and/or a dielectric layer. For example, the insulation layer 102 is disposed on the base substrate 100 and covers the sidewalls of the conductive layer 101 and the surface thereof at a side away from the base substrate 100; the dummy layer M1 is disposed on a side of the insulation layer 102 away from the base substrate 100, and is electrically isolated from the conductive layer 101 by the insulation layer 102; the insulation layer 105 is disposed on a side of the insulation layer 102 away from the base substrate 100, and covers the sidewalls of the dummy layer M1 and the surface thereof at a side away from the base substrate 100; the conductive layer 106 is disposed on a side of the insulation layer 105 away from the base substrate 100 and is electrically isolated from the dummy layer M1 by the insulation layer 105; the dielectric layer 107 is disposed on a side of the insulation layer 105 away from the base substrate 100, and covers the sidewalls of the conductive layer 106 and the surface thereof at a side away from the base substrate 100; and the hydrophobic layer 108 is disposed on a side of the dielectric layer 107 away from the base substrate 100, and is isolated from the conductive layer 106 by the dielectric layer 107.

In some embodiments, the base substrate 100 may be or include a glass substrate; the conductive layers 101 and 106 may include the same or different conductive materials, for example, the conductive layers 101 and 106 may each include a transparent conductive material, such as indium tin oxide (ITO), indium zinc oxide (IZO), etc. The thicknesses of conductive layers 101 and 106 may be the same or different, and may each be, for example, greater than or equal to 52 nanometers (nm).

The material of the dummy layer M1 is different from the materials of the conductive layers 101 and 106. For example, the dummy layer M1 is opaque and may include an opaque conductive material, such as a metal material; for example, the dummy layer M1 may include a metal material such as molybdenum (Mo), molybdenum aluminum (Mo/Al) alloy, or the like. The thickness of the dummy layer M1 may be the same as or different from the thickness of the conductive layer 101 or 106; and the thickness of the dummy layer M1 may be, for example, greater than or equal to 52 nm.

The insulation layers 102 and 105 may each be selected from insulation materials such as silicon oxide, silicon nitride, silicon oxynitride, etc., and may each have a thickness greater than or equal to 300 nm; for example, in some examples, the insulation layers 102 and 105 are both made of silicon nitride material, and each has a thickness of 600 nm, but the present disclosure is not limited thereto.

The dielectric layer 107 may include an inorganic dielectric material, an organic dielectric material, or a combination thereof; for example, the dielectric layer 107 may be selected from one or more of silicon oxide, silicon nitride, SU8, and polyimide (PI); in some examples, the dielectric layer 107 is made of PI material and may have a thickness of about 28 microns (m), but the present disclosure is not limited thereto.

The hydrophobic layer 108 may include a fluorine-containing material, such as Teflon, CYTOP, etc.; the thickness of the hydrophobic layer 108 may be greater than or equal to 100 nm, and the coverage range of the hydrophobic layer 108 may be approximately the same as the coverage range of the dielectric layer 107. That is, the orthographic projection of the hydrophobic layer 108 on the base substrate 100 may approximately coincide with the orthographic projection of the dielectric layer 107 on the base substrate 100. For example, the dielectric layer 107 and the hydrophobic layer 108 cover and across the entire surface of the underlying material layers over the base substrate 100.

Continuing to refer to FIG. 1A to FIG. 1C, in some embodiments, the second substrate S2 includes a base substrate 200, a conductive layer 206 and a hydrophobic layer 208. The conductive layer 206 is disposed on a side of the base substrate 200, and the hydrophobic layer 208 is disposed on a side of the conductive layer 206 away from the base substrate 200. The droplet channel DC is, for example, defined between the hydrophobic layer 108 of the first substrate S1 and the hydrophobic layer 208 of the second substrate S2.

The base substrate 200 may be or include a glass substrate. The material and thickness of the conductive layer 206 may be the same as or different from the materials of the conductive layers 101 and 106. For example, the conductive layer 206 may include a conductive material such as ITO, IZO, etc., and may have a thickness greater than or equal to 52 nm and a sheet resistance of less than about 150 ohms per square (Ω/□). The candidate materials and thickness ranges of the hydrophobic layer 208 are substantially the same as those of the hydrophobic layer 108 and will not be described again here.

In some embodiments, the conductive layer 206 extends continuously on a side of the base substrate 200, for example, covering the entire surface of the base substrate 200 at a side close to the first substrate S1. The first substrate S1 may include a ground electrode G, which may be included in the conductive layer 106 and electrically isolated from other conductive structures such as driving electrodes in the conductive layer 106; a conductive structure (not shown) is provided between the first substrate S1 and the second substrate S2 to electrically connect the ground electrode G of the first substrate S1 to the conductive layer 206 of the second substrate S2, so that the conductive layer 206 is grounded; and the conductive structure may be or include conductive foam, and for example, has a thickness of about 500 μm. The ground electrode G and the conductive structure are, for example, in the peripheral region R2.

In some embodiments, a frame sealing adhesive 300 is provided between the first substrate S1 and the second substrate S2, and the frame sealing adhesive 300 is used for the cell assembly and cell thickness support of the first substrate and the second substrate; at least a part of the frame sealing adhesive 300 is arranged along the periphery of the first substrate S1 and the second substrate S2, and surrounds the body region R1 of the microfluidic chip; and the droplet channel DC is, for example, defined by the surfaces of the first substrate S1 and the second substrate S2 facing each other, and the sidewalls of the frame sealing adhesive 300. For example, the frame sealing adhesive 300 may have a width of about 2 mm and a thickness of about 500 μm, and the spacing between the frame sealing adhesive 300 and the ground electrode G (for example, in the direction D2) may be greater than 200 μm. The edge of a portion of the frame sealing adhesive 300 away from the peripheral region R2 and the bonding region R3 may be substantially aligned with edges of the first substrate and the second substrate in a direction perpendicular to the main surface of the substrate.

In some embodiments, the second substrate S2 includes one or more liquid adding holes 201 that penetrate through the second substrate S2 and are in communication with the droplet channel DC; the liquid adding holes 201 are used to add an environmental solution (for example, oil) into the droplet channel DC, and may also be referred to as oiling holes; in some embodiments, four oiling holes 201 are provided at four corners of the body region R1, but the present disclosure is not limited thereto.

In some embodiments, the second substrate S2 further includes sample inlets 202a, 202b, and 202c; and the sample inlets 202a, 202b, and 202c penetrate through the second substrate S2 and are in communication with the droplet channel DC for adding sample solutions, reaction solutions, etc., into the droplet channel DC. It should be understood that the solutions applied through the sample inlets 202a, 202b, 202c are insoluble in the environmental solution added through the liquid adding hole 201. The sample inlets 202a, 202b, and 202c are respectively provided at positions corresponding to the liquid generation modules B-1, B-2, and B-3. It should be understood that in different steps of the biochemical process, each sample inlet may also be reused as a sample outlet for taking out the target substance from the opening.

Referring to FIG. 1A, in some embodiments, an alignment mark H, such as a cross mark, may be provided on the first substrate S1, but the present disclosure is not limited thereto. The alignment mark H may be an alignment mark used for chip cutting and dielectric layer attachment.

Still referring to FIG. 1A to FIG. 1C, in some embodiments, each of the droplet generation structure GS, the droplet receiving module RM, and the interconnection module IM in the digital microfluidic chip 500 may include one or more electrode array regions and a stacked structure (i.e., a portion of the stacked structure of the first substrate, the second substrate and the droplet channel) overlapping with the electrode array region in a direction perpendicular to the main surface of the chip, and each electrode array region may include a plurality of driving electrode structures DS spaced apart from each other; the plurality of driving electrode structures DS are located in the first substrate S1, and each driving electrode structure DS includes a driving electrode DE, a dummy electrode 103a, and a conductive connection pad CP, which are respectively located in the conductive layer 106, the dummy layer M1, and the conductive layer 101. As shown in FIG. 1A, the planar shape of each driving electrode structure DS in a top view is approximately rectangular or square, and the areas of the driving electrode structures DS in different modules may be different. The area of the driving electrode structure DS is approximately equal to the area of its driving electrode DE.

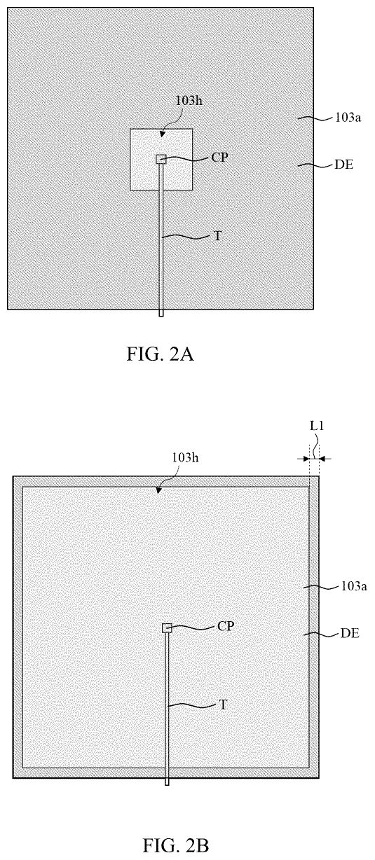

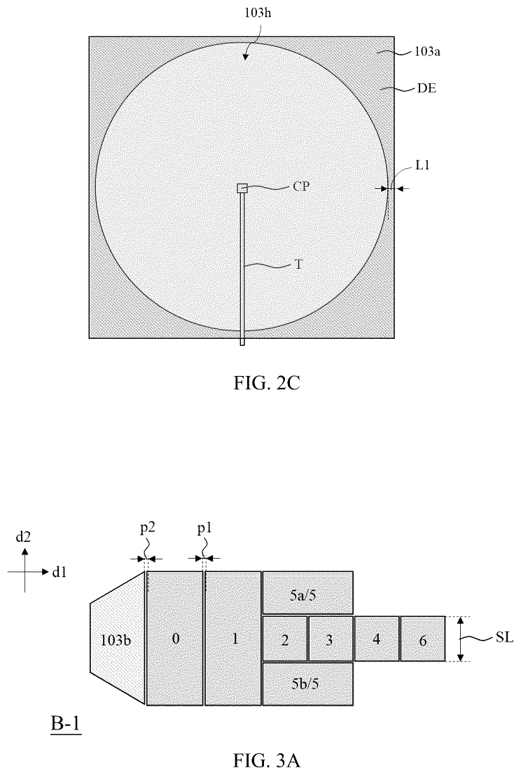

FIG. 2A to FIG. 2C illustrate top views of a driving electrode structure DS in a digital microfluidic chip according to some embodiments of the present disclosure.

Referring to FIG. 1C and FIG. 2A, in some embodiments, the peripheral contours of the driving electrode DE and the dummy electrode 103a may substantially coincide with each other, that is, the peripheral contour of the orthographic projection of the driving electrode DE on the main surface of the base substrate 100 may substantially coincide with the peripheral contour of the orthographic projection of the dummy electrode 103a on the main surface of the base substrate 100. In other words, the outer sidewall of the driving electrode DE and the outer sidewall of the dummy electrode 103a are substantially aligned with each other in a direction perpendicular to the main surface of the base substrate 100. The dummy electrode 103a has an opening 103h, and the driving electrode DE passes through the opening 103h to be connected to the conductive connection pad CP. For example, the driving electrode DE is electrically connected to the conductive connection pad CP through the conductive via V, and the conductive connection pad CP is connected to the conductive wire T that is connected to the bonding electrode. The conductive via V fills the via holes of the insulation layers 102 and 105 and is located in the opening 103h of the dummy electrode 103a. A portion of the insulation layer 105 is located between the dummy electrode 103a and the conductive via V, such that the dummy electrode 103a and the conductive via V are spaced apart and electrically isolated from each other.

In some embodiments, the use of transparent conductive materials for the conductive layers 101 and 106 can avoid the absorption of visible light by the microfluidic chip to the greatest extent, thereby improving the optical performance of the chip, so that the microfluidic chip of the present embodiment can meet the requirements of fluorescence detection and the like. In some embodiments, the insulation layer is also transparent. However, if both the driving electrode and the insulation layer are made of transparent materials, the position of the driving electrode cannot be determined through optical methods; thus, in the case where the position of the driving electrode cannot be accurately determined, this will make it impossible to determine whether there is an error in the sample adding process during the sample adding operation (for example, when manually adding the sample through a pipette is required), which will be unfavorable for the use of the chip; On the other hand, when performing processes such as cell assembly or cutting on the first substrate and the second substrate, there is a need for an opaque pattern other than the alignment mark structures to complete image capture, thereby ensuring the accuracy of the chip while realizing automated production. In addition, when routing a plurality of conductive wires in the conductive layer 101, there may be a plurality of conductive wires under the driving electrodes (as shown in FIG. 1A), and this plurality of conductive wires may include conductive wires corresponding to other driving electrodes, which may lead to signal crosstalk between driving electrodes and affect the manipulation of droplets. For example, if there is a conductive wire (also referred to as a crosstalk wire) connected to other driving electrodes under a certain driving electrode, this conductive wire forms a capacitor with the driving electrode. When the conductive wire is powered on, a coupling voltage will appear on the driving electrode due to the coupling effect of the capacitor, causing signal crosstalk, and further affecting the manipulation of droplets.

The embodiments of the present disclosure can solve the above problem by providing the dummy electrode 103a. Because the dummy electrode 103a is opaque and coincides with the peripheral contour of the driving electrode DE, the position of the dummy electrode 103a can be determined by optical methods, thereby accurately determining the position of the corresponding driving electrode DE. Moreover, there is an opening 103h in the middle of the dummy electrode 103a, so the optical performance of the location of the droplet can also be ensured. On the other hand, the dummy electrode 103a is made of metal material and is electrically floating or grounded, which can effectively shield the signals of the conductive layer 101, avoiding or reducing signal crosstalk between different driving electrodes, that is, the dummy electrode 103a can shield the crosstalk to the driving electrode DE from other conductive wires in the conductive layer 101 that are not connected to the corresponding driving electrode DE. In this way, the accuracy and reliability of droplet manipulation by the driving electrodes in the microfluidic chip can be improved.

In some embodiments, the size and dimensions of the opening of the dummy electrode 103a may be adjusted according to product design and requirements. For example, as shown in FIG. 2A, in some examples, the size of the opening 103h of the dummy electrode 103a corresponds to the minimum field diameter of a fluorescence microscope. For example, the opening 103h may be a square with a side length of about 50 μm. In this example, the opening range of the dummy electrode 103a is controlled to the minimum, thereby increasing the area of the dummy electrode. Thus, signal shielding of the crosstalk wires in the conductive layer 101 by the dummy electrode can be maximized, while simultaneously satisfying the optical performance requirements of the driving structure.

Referring to FIG. 2B, in other examples, the size of the opening 103h of the dummy electrode 103a is set relatively large to maximize the transparent range of the driving electrode structure DS while ensuring that the position of the driving electrode can be determined with the naked eye, thereby satisfying fluorescence detection requirements with a wide field of view. For example, the boundary of the opening 103h of the dummy electrode 103a is shifted inward approximately 100 microns from the boundary of the driving electrode DS. In other words, the distance L1 between the outer sidewall of the dummy electrode 103a and the inner sidewall defining the opening 103h is approximately 100 microns.

Referring to FIG. 2C, in some embodiments, the shape of the opening 103h of the dummy electrode 103a may be circular, and the minimum distance from the boundary of the opening 103h to the boundary of a corresponding driving electrode DE, in a direction parallel to the main surface of the base substrate, needs to be greater than or equal to 100 microns to ensure visibility to the human eye. In other words, the distance L1 between the outer sidewall of the dummy electrode 103a and the inner sidewall thereof defining the opening 103h is greater than or equal to 100 microns.

It should be understood that the above-mentioned shape and size of the opening of the dummy electrode 103a are only for illustration, and the present disclosure is not limited thereto. The shape and size of the dummy electrode may be designed according to actual application requirements; for example, when a large field of view for fluorescence detection is required, the embodiment of FIG. 2B is a preferred embodiment; and when there is no requirement for the field of view of fluorescence detection, the embodiment of FIG. 2A is a preferred embodiment.

The present embodiment takes the cross-sectional structure at the interconnection module IM as an example to illustrate the stacked structure of the microfluidic chip. It should be understood that other modules such as the droplet generation module in the microfluidic chip have similar stacked structures, and having the same features as described above with reference to FIG. 1C and FIG. 2A-FIG. 2C.

In some embodiments, as shown in FIG. 1A, the plurality of droplet generation modules may include at least one droplet generation module B-1, at least one droplet generation module B-2 and at least one droplet generation module B-3, which are respectively configured to generate the first droplet, the second droplet, and the third droplet. The droplet generation modules B-1, B-2, and B-3 may also be referred to as a first droplet generation module, a second droplet generation module, and a third droplet generation module, respectively. It should be understood that the numbers of the droplet generation modules may be designed and adjusted according to actual application requirements, and may be the same as or different from each other. Each droplet generation module can achieve the addition, removal, storage of a sample, as well as the generation of droplets.

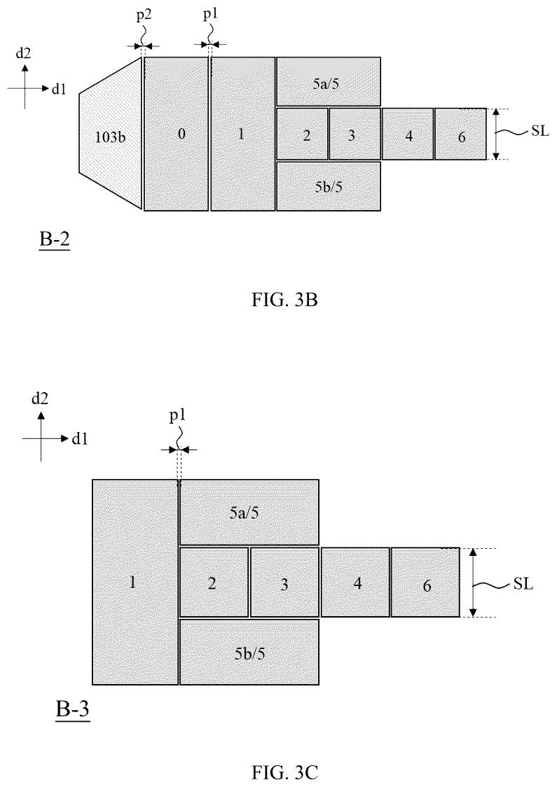

FIG. 3A to FIG. 3C respectively illustrate top views of droplet generation modules B-1, B-2, and B-3 according to some embodiments of the present disclosure.

Referring to FIG. 3A to FIG. 3C, in some embodiments, each of droplet generation modules B-1, B-2, B-3 may include driving electrodes 1, 2, 3, 4, 5, 6. The driving electrodes 1, 2, 3, 4, 5 and 6 may also be respectively referred to as a first driving electrode, a second driving electrode, a third driving electrode, a fourth driving electrode, a fifth driving electrode and a sixth driving electrode. It should be understood that each driving electrode in each droplet generation module corresponds to a driving electrode structure DS as shown in FIG. 1C, and for the sake of simplicity, only the driving electrodes on the upper layer are shown in FIG. 3A to FIG. 3C, the dummy electrodes, conductive vias, conductive connection pads and other structures between the corresponding driving electrodes and the base substrate are not shown in FIG. 3A to FIG. 3C, and the corresponding structures can refer to the content described above regarding FIG. 1C, and FIG. 2A to FIG. 2C. The relative positional relationship between the driving electrodes of each droplet generation module described herein may also be interpreted as the relative positional relationship between the corresponding driving electrode structures.

In some embodiments, in each of the droplet generation modules B-1, B-2, and B-3, the driving electrodes 2, 3, 4, 6 are sequentially arranged in a first sub-direction d1 and have the same first area as each other; and the driving electrode 6 is located at an end of the corresponding droplet generation module and is configured to be connected (e.g., directly connected) to a driving electrode in another module or region. The driving electrode 5 includes two driving sub-electrodes (also be referred to as fifth driving sub-electrodes) 5a and 5b that are electrically connected to each other, the driving sub-electrodes 5a and 5b are arranged on opposite sides of the driving electrodes 2 and 3 in a second sub-direction d2 perpendicular to the first sub-direction d1, and each driving sub-electrode 5a/5b has a second area; the driving electrode 1 is arranged on a side of the driving electrode 2 away from the driving electrode 3 in the first sub-direction d1, and connected to the driving electrodes 2 and 5, and the driving electrode 1 may have a third area; for example, the first area is smaller than the second area, and the second area is smaller than the third area. In a plurality of driving electrodes, adjacent driving electrodes are spaced apart from each other. It should be noted that in the present disclosure, two or more driving electrodes being directly connected to each other means that the two or more driving electrodes are immediately adjacent to each other, and two or more driving electrodes being connected to each other includes the case where the driving electrodes are immediately adjacent to each other or the driving electrodes are spaced apart from each other by other electrode(s) therebetween; and the connection does not limit the two or more driving electrodes to be physically or electrically connected to each other, unless it is clearly defined as a physical connection or electrical connection. The driving electrodes are connected to each other, for example, so that the droplets can be stored on the driving electrodes and/or the droplets can be driven to move between the driving electrodes.

In some embodiments, in each of droplet generation modules B-1 and B-2, an additional driving electrode 0 may also be included. The additional driving electrode 0 is located on a side of the driving electrode 1 away from the driving electrode 2 in the first sub-direction d1 and may have the third area substantially the same as the driving electrode 1. In some embodiments, an additional dummy electrode may also be included in one or more droplet generation modules. For example, in each of the droplet generation modules B-1 and B-2, an additional dummy electrode 103b may be further included, and the additional dummy electrode 103b is located on a side of the additional driving electrode 0 away from the driving electrode 1 in the first sub-direction d1. In some embodiments, the additional dummy electrode 103b is located in the dummy layer M1 shown in FIG. 1C, that is, provided in the same layer as the dummy electrode 103a in each driving electrode structure DS. In the present disclosure, two patterns “provided in the same layer” means that the two patterns are formed from the same material layer through the same patterning process. In some embodiments, no corresponding driving electrode is provided on a side of the additional dummy electrode 103b away from the base substrate 100, that is, the orthographic projection of the additional dummy electrode 103b on the base substrate 100 does not overlap with the orthographic projection of a driving electrode and/or a conductive layer 106 in which the driving electrode is located on the base substrate 100.

In some embodiments, the additional dummy electrode 103b is served as an alignment mark for a sample inlet of the second substrate to mark the location of a corresponding droplet generation module, so as to facilitate sample addition. For example, as shown in FIG. 1A, for the droplet generation modules B-1 and B-2 each having the additional dummy electrode 103b, the orthographic projections of the additional dummy electrodes 103b of the droplet generation modules B-1 and B-2 on the main surface of the first substrate S1 respectively overlap (either partially or completely) with the orthographic projections of the corresponding sample inlets 202a and 202b on a main surface of the first substrate S1 facing the second substrate S2.

In some embodiments, a droplet generation module without additional dummy electrodes, such as the droplet generation module B-3, may be included in the microfluidic chip. For this type of droplet generation module without additional dummy electrodes, its corresponding sample inlet may be provided at a position adjacent to but not overlapping its driving electrode. For example, for the droplet generation module B-3 without additional dummy electrodes, the orthographic projection of the corresponding sample inlet 202c on the main surface of the first substrate S1 is located at a position adjacent to the orthographic projection of the driving electrode 1 of the droplet generation module B-3 on the main surface of the first substrate S1, but does not overlap with the orthographic projection of the driving electrode 1 on the main surface of the first substrate S1.

In some embodiments, the volume of a droplet that can be accommodated and driven by a driving electrode (also be referred to as a main electrode) with the smallest area in each droplet generation module is approximately equal to the volume of a single droplet generated by a corresponding droplet generation module. For example, the droplet generation modules B-1, B-2, and B-3 are respectively configured to generate droplets with the first volume (for example, 1 μL), the second volume (for example, 2 μL), and the third volume (for example, 5 μL), then the volume of a droplet that can be accommodated and driven by the driving electrode with the smallest area (such as driving electrodes 2, 3, 4, 6) in the droplet generation module B-1 is approximately equal to the first volume; the volume of a droplet that can be accommodated and driven by the driving electrode with the smallest area (such as driving electrodes 2, 3, 4, 6) in the droplet generation module B-2 is approximately equal to the second volume; and the volume of a droplet that can be accommodated and driven by the driving electrode with the smallest area (such as driving electrodes 2, 3, 4, 6) in the droplet generation module B-3 is approximately equal to the third volume.

In some examples, as shown in FIG. 3A to FIG. 3C, in each droplet generation module, the driving electrodes 2, 3, 4, and 6 may all be square electrodes, and each has a side length SL. Each driving sub-electrode of the driving electrode 5 may be a rectangular electrode, and the rectangular electrode has a long edge along the first sub-direction d1 and a short edge along the second sub-direction d2. The length of the long edge is approximately equal to twice the side length SL of the square electrode within the same generation module, and the length of the short edge is approximately equal to the side length SL of the corresponding square electrode. The driving electrode 1 or 0 may be a rectangular electrode and have a short edge along the first sub-direction d1 and a long edge along the second sub-direction d2, the length of the short edge is approximately equal to the side length SL of the corresponding square electrode, and the length of the long edge is approximately equal to three times the side length SL of the corresponding square electrode. In some embodiments, the additional dummy electrode 103b may have a different shape from the driving electrode, for example, it may be trapezoidal. In some examples, the additional dummy electrode 103b may be in a shape of an isosceles trapezoid and have a short edge (or called upper base) and a long edge (or called lower base) parallel to each other extending along the second sub-direction d2, the length of the short edge is approximately equal to 1.5 times the side length SL of the square electrode in the same generation module, and the length of the long edge is approximately equal to three times the side length SL of the corresponding square electrode. In the present disclosure, two dimension features (such as side lengths, areas, widths, lengths, volumes, etc.) being approximately equal may include that the two dimension features are completely equal, and may also include the case where the difference between each dimension feature and the average value of the two dimension features is less than 5% to 20% of the average value, for example, less than 5%, 10%, 15%, or 20% of the average value. For example, the side length of the rectangular electrode being approximately equal to the side lengths of two square electrodes may include the case where the side length of the rectangular electrode is equal to the sum of the side lengths of the two square electrodes and the spacing between the two square electrodes. It should be understood that the shapes and sizes of the above-mentioned driving electrodes, additional driving electrodes and/or additional dummy electrodes are only for illustration, and the present disclosure is not limited thereto. For example, in some embodiments, the shape of the additional dummy electrode may also be circular, square, etc.

In some embodiments, adjacent driving electrodes are spaced apart from each other by a spacing p1, and the spacings between different adjacent driving electrodes in the plurality of driving electrodes of respective modules in the chip may be approximately equal to each other. The additional dummy electrode 103b and the adjacent driving electrode 0 are spaced apart from each other by a spacing p2, and the spacings p2 between different adjacent electrodes in respective modules may be approximately equal to each other. The spacings p1 and p2 may differ from each other, for example, the spacing p2 is greater than the spacing p1. In some examples, the spacing p1 between adjacent driving electrodes is about 25 μm, and the spacing p2 between the additional dummy electrode 103b and the adjacent driving electrode 0 is about 200 μm, but the present disclosure is not limited thereto.

In some embodiments, the droplet generation modules B-1, B-2, and B-3 are respectively configured to generate droplets of different volumes, and the areas of the corresponding driving electrodes in the droplet generation modules B-1, B-2, and B-3 are different. For example, a first area, a second area, and a third area of corresponding driving electrodes in the droplet generation module B-1 are respectively smaller than a first area, a second area, and a third area of corresponding driving electrodes in the droplet generation module B-2, and the first area, the second area, and the third area of the corresponding driving electrodes in the droplet generation module B-2 are respectively smaller than a first area, a second area, and a third area of corresponding driving electrodes in the droplet generation module B-3. The area of each driving electrode in each droplet generation module may be set according to the volume of a single droplet that the droplet generation module is configured to generate. For example, the volume V of a droplet in a digital microfluidic chip may be estimated by the following formulas:

$$V = π δ {a}^{2} + 2 π {( \frac{δ}{2 cos θ} )}^{2} [ {af}_{1} ( θ ) + \frac{δ}{2} {f}_{2} ( θ ) ]$$

$${f}_{1} ( θ ) = θ - \frac{π}{2} + \frac{sin ( 2 θ - π )}{2} + 2 sin θ cos θ$$

$${f}_{2} ( θ ) = tan θ [ θ - \frac{π}{2} + \frac{sin ( 2 θ - π )}{2} + 2 cos θ ] - \frac{1}{3} {cos}^{2} θ + {( 1 - sin θ )}^{2}$$

wherein θ is the contact angle of the droplet, δ is the cell thickness of the digital microfluidic chip, that is, the distance between the first substrate S1 and the second substrate S2, and a is the radius of the solid-liquid contact surface of the droplet. For example, the radius a of the solid-liquid contact surface of the droplet is calculated based on the volume of a single droplet that the droplet generation module is configured to generate. The side length SL and area of the square electrode with the smallest area in the droplet generation module can be derived from the value a, and then the corresponding dimensions of other driving electrodes and/or additional dummy electrodes in the droplet generation module can be extrapolated.

For example, the droplet generation modules B-1, B-2, and B-3 are respectively configured to generate droplets with volumes of 1 μL, 2 μL, and 5 μL. By using the above-mentioned formulas, it can be calculated that the side length SL of each of the square driving electrodes 2, 3, 4, and 6 within the droplet generation module B-1 is 1084 μm, the side length SL of each of the square driving electrodes 2, 3, 4, and 6 within the droplet generation module B-2 is 1550 μm, and the side length SL of each of the square driving electrodes 2, 3, 4, and 6 within the droplet generation module B-3 is 2474 μm. The dimensions of the other electrodes in each droplet generation module may be determined according to the multiples of the side length SL described above. In some embodiments, due to the relatively large size of the droplet generation module B-3, the additional driving electrode 0 and the additional dummy electrode 103b are omitted in the droplet generation module B-3, which is beneficial to saving chip area, but the present disclosure is not limited thereto. In some other embodiments, the droplet generation module B-3 may also include an additional driving electrode 0 and an additional dummy electrode 103b; alternatively, one or more of the droplet generation modules B-1 and B-2 may also omit the additional driving electrode 0 and the additional dummy electrode 103b, as long as each droplet generation module can generate the droplets of corresponding volumes.

When using a digital microfluidic chip to perform a sample adding operation, use a pipette to inject the required solution from a corresponding sample inlet, and then apply a driving signal to a corresponding driving electrode in the droplet generation module to complete the sample adding operation and the generation of droplet. For example, for each of the droplet generation modules B-1 and B-2, after injecting the sample solution from the corresponding sample inlet, the driving electrodes 0 and 1 are turned on, and then the driving electrodes 0 and 1 are turned off, and the driving electrodes 2, 3, and 5 are turned on to complete the sample adding operation. After completing the sample adding operation, the droplet generation operation includes: first turning on the driving electrodes 2, 3, 4, and 6 so that the droplet moves to the driving electrodes 2, 3, 4, and 6, then turning off the driving electrode 4 and turning on the driving electrode 5, causing the droplet to split into a droplet of a target volume on the driving electrode 6 and a droplet of the remaining volume on the driving electrodes 2, 3, and 5, thereby achieving the generation of the droplet of the target volume. The droplet of the target volume may subsequently be driven and moved to other modules through the interconnection module. For the droplet generation module B-3, after injecting the sample solution from the sample inlet, turning on the driving electrodes 1, 2, and 3, then turning off the driving electrode 1, and turning on the driving electrode 5 to complete the sample adding operation. The droplet generation operation of the droplet generation module B-3 is substantially the same as that of the above-mentioned droplet generation modules B-1 and B-2, and will not be described again here. It should be understood that in the present disclosure, “turning on the driving electrode” means applying a driving signal to the driving electrode, that is, supplying power to the driving electrode; and “turning off the driving electrode” means not applying a driving signal to the driving electrode, that is, the driving electrode is not supplied with power or powered off.

In the above embodiments, each droplet generation module is configured to generate the droplet of a single volume type, but the present disclosure is not limited thereto. In some other embodiments, one or more droplet generation modules in the digital microfluidic chip are configured to generate droplets of different volume types. For example, the digital microfluidic chip may include one or more fourth droplet generation modules, and each fourth droplet generation module is configured to generate at least two of the first droplet with the first volume, the second droplet with the second volume, and the third droplet with the third volume.

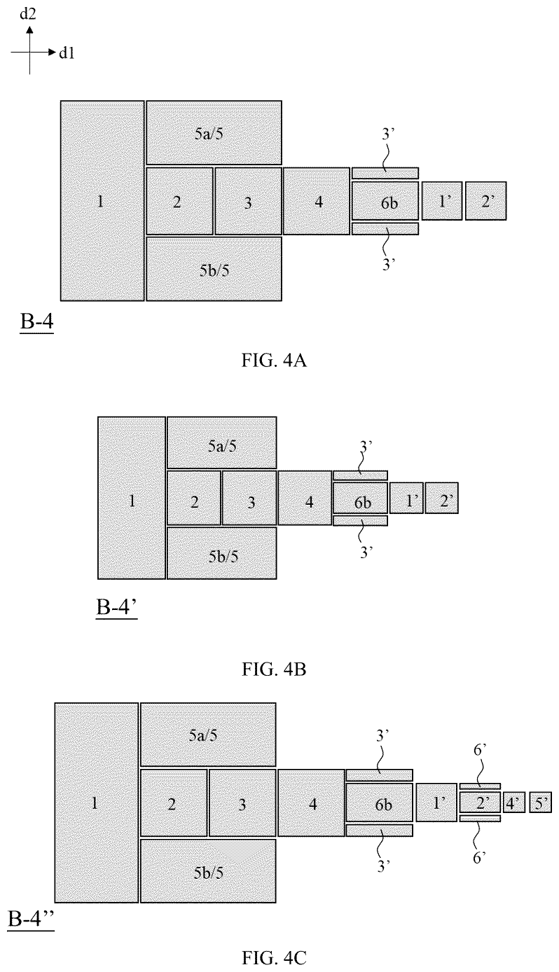

FIG. 4A to FIG. 4C illustrate schematic plan views of a droplet generation module (e.g., a fourth droplet generation module) according to some other embodiments of the present disclosure.

Referring to FIG. 4A, in some embodiments, the droplet generation module B-4 may include driving electrodes 1, 2, 3, 4, 5, 6b, an additional driving electrode 1′, an additional driving electrode 2′, and two additional driving electrodes 3′. The additional driving electrodes 1′, 2′, and 3′ may be referred to as a first additional driving electrode, a second additional driving electrode, and a third additional driving electrode, respectively. In the droplet generation module B-4, the relative positional relationship of the driving electrodes 1-5 is substantially the same as the relative positional relationship of the driving electrodes 1-5 in the aforementioned droplet generation module.

For example, the driving electrodes 2, 3, 4, 6b, 1′, 2′ are arranged sequentially in the first sub-direction d1, and the two additional driving electrodes 3′ are arranged on opposite sides of the driving electrode 6b in the second sub-direction d2 perpendicular to the first sub-direction d1. Two driving sub-electrodes 5a and 5b of the driving electrode 5 are arranged on opposite sides of the driving electrodes 2 and 3 in the second sub-direction d2, and the driving electrode 1 is arranged on a side of the driving electrodes 2 and 5 away from the driving electrode 4 in the first sub-direction d1. In the droplet generation module B-4, the additional driving electrode 2′ is located at an end and is connected to a driving electrode in other modules or regions such as the interconnection module, for example, may be connected to the droplet driving module IM2 in the interconnection module.

In some embodiments, the driving electrodes 2, 3, and 4 are square electrodes and have the same first body area as each other; the additional driving electrodes 1′ and 2′ may have the same second body area as each other; in this embodiment, the driving electrode 6b is a rectangular electrode, and the sum of the areas of the driving electrode 6b and two additional driving electrodes 3′ is approximately equal to the first body area; and the second body area is smaller than the first body area. In some embodiments, the first body area and the second body area respectively correspond to the volumes of two types of droplets that the droplet generation module B-4 is configured to generate. Here, the sum of the areas of the driving electrode 6b and two additional driving electrodes 3′ being approximately equal to the first body area may include the case that the sum of the areas of the driving electrode 6b and two additional driving electrodes 3′ is completely equal to the first body area, and may also include the case that the sum of the areas of the driving electrode 6b and two additional driving electrodes 3′, and the areas of gap regions between the driving electrode 6b and the two additional driving electrodes 3′ is equal to the first body area.

In the fourth droplet generation module B-4, the driving electrode 1 and the additional driving electrode 1′ are electrically connected to each other, the driving electrode 2 and the additional driving electrode 2′ are electrically connected to each other, and the driving electrode 3 and the additional driving electrode 3′ are electrically connected to each other. The driving electrodes that are electrically connected to each other receive the same driving signal, that is, they are powered on simultaneously, or powered off simultaneously.

For example, in some embodiments, the droplet generation module B-4 is configured to generate the second droplet having the second volume and the third droplet having the third volume; in this embodiment, the volume of a droplet that can be accommodated and driven by each driving electrode (i.e., additional driving electrode 1′, 2′) having the second body area is approximately equal to the second volume of the second droplet, and the volume of a droplet that can be accommodated and driven by a single driving electrode (i.e., driving electrode 2, 3, 4) or a combination of driving electrodes (i.e., a combination of the driving electrode 6 and two additional driving electrodes 3′) having the first body area is approximately equal to the third volume of the third droplet. For example, the droplet generation module B-4 may be further configured to generate an additional droplet, and the volume of the additional droplet is approximately equal to the third volume minus the second volume.

For example, the dimensions of driving electrodes 1-5 may be the same as the dimensions of corresponding driving electrodes in the droplet generation module B-3 shown in FIG. 3C. The driving electrode 6b has a long edge along the first sub-direction d1 and a short edge along the second sub-direction d2, the length of the long edge is approximately equal to the side length of each of the square electrodes 2-4, and the length of the short edge is approximately equal to the side length of the electrode 1′ or 2′. For example, in the case where the second volume and the third volume are respectively 2 μL and 5 μL, the length of the long edge of the driving electrode 6′ is approximately 2474 μm, and the length of the short edge of the driving electrode 6′ is approximately 1550 μm. The driving electrode 3′ has a long edge along the first sub-direction d1 and a short edge along the second sub-direction d2, the length of the long edge is approximately 2474 μm, and the length of the short edge is approximately 437 μm. The additional driving electrodes 1′ and 2′ may be square electrodes and each have the same size as the square electrode in the droplet generation module B-2. For example, the side length of each of the additional driving electrodes 1′ and 2′ is approximately 1550 μm.

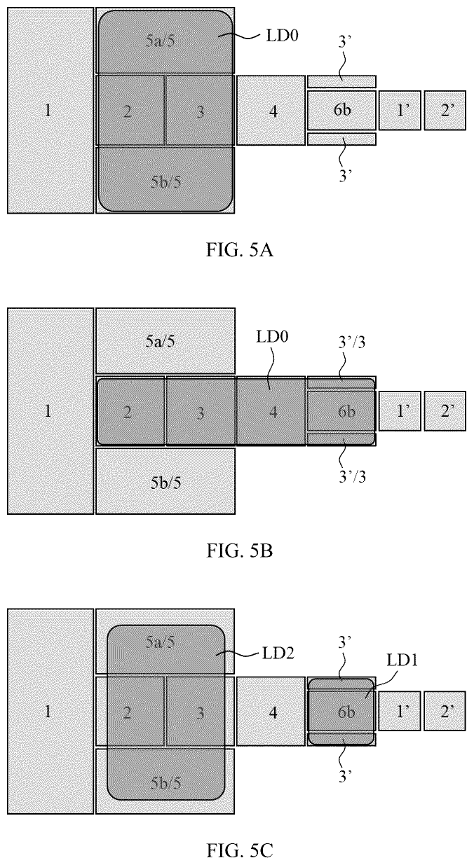

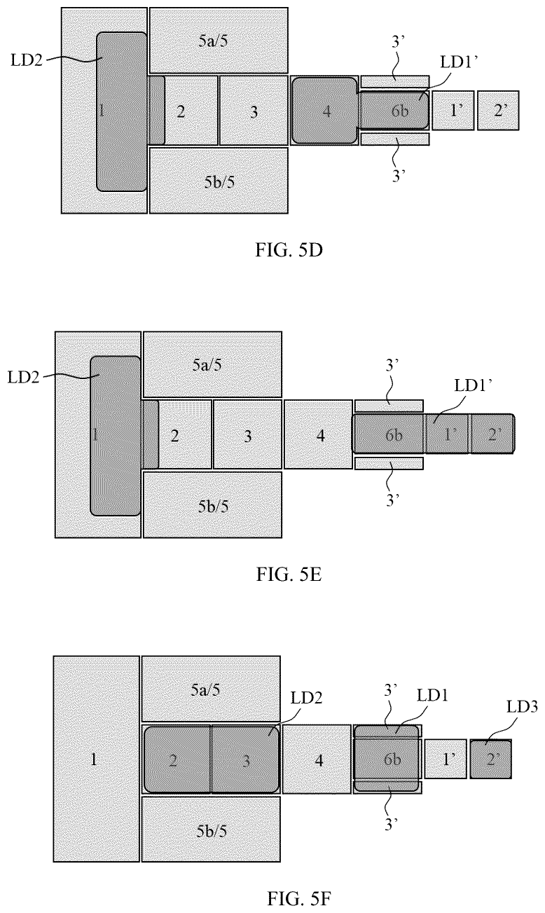

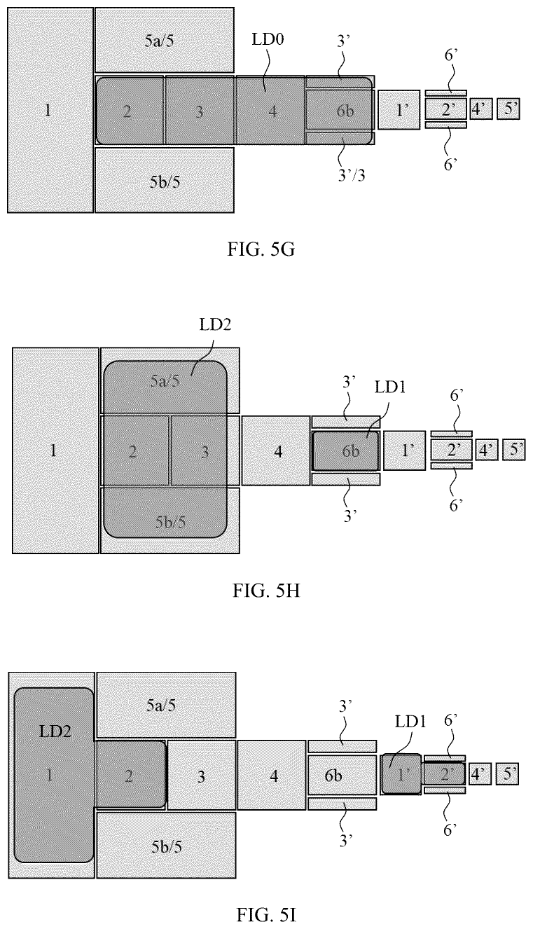

In this embodiment, the process of generating a droplet with a second volume (for example, 2 μL) or a third volume (for example, 5 μL) by the droplet generation module B-4 is illustrated in FIG. 5A to FIG. 5F.

Referring to FIG. 5A to FIG. 5C, in some embodiments, after completing the sample adding operation, the driving electrodes 2, 3, and 5 are powered on, thereby driving the droplet to move to the driving electrodes 2, 3, and 5, and the shape of the droplet LD0 is shown in FIG. 5A. Next, the driving electrode 5 is powered off and the driving electrodes 4 and 6b are powered on; that is, the driving electrodes 2, 3, 4, 6b and the additional driving electrode 3′ are in an open state, thereby driving the droplet LD0 to move to the open driving electrodes, the state of driving the droplet LD0 in this step is shown in FIG. 5B. After that, the driving electrode 4 is powered off and the driving electrode 5 is powered on, so that the droplet LD0 is driven by the driving electrodes 3′, 6b and the driving electrodes 2, 3, 5 to move toward opposite directions and split into droplets LD1 and LD2, and the droplet LD1 is the target droplet with the third volume (for example, 5 μL).

Referring to FIG. 5A, FIG. 5B and FIG. 5D to FIG. 5F, in some embodiments, the driving electrodes 2, 3, and 5 are powered on to drive the droplet to move to the driving electrodes 2, 3, and 5, and the shape of the droplet LD0 is shown in 5A. Next, the driving electrode 5 is powered off and the driving electrodes 4 and 6b are powered on; that is, the driving electrodes 2, 3, 4, 6b and the additional driving electrode 3′ are in an open state, thereby driving the droplet LD0 to move to the open driving electrodes, the state of driving the droplet LD0 in this step is shown in FIG. 5B. Referring to FIG. 5B and FIG. 5D, the driving electrodes 2 and 3 are powered off, and the driving electrode 1 is powered on, that is, the driving electrodes 1, 4, and 6b are in an open state, so that the droplet LD0 is respectively driven by the driving electrodes 4, 6b, and the driving electrode 1 to move toward opposite directions and split into droplets LD1′ and LD2. It should be understood that FIG. 5D shows the intermediate process of droplet splitting, so a portion of the droplet LD2 is on the driving electrode 2, and after the splitting is completed, the droplet LD2 will move to the driving electrode 1 as a whole.

Referring to FIG. 5D to FIG. 5E, thereafter, the driving electrode 4 is powered off, and the additional driving electrode 2′ and the driving electrode 2 are powered on; that is, the driving electrodes 1, 2, 6b and the additional driving electrodes 1′ and 2′ are in an open state; therefore, the droplet LD1′ moves onto the driving electrodes 1′, 2′, and 6b, and the droplet LD2 partially moves from the driving electrode 1 to the driving electrode 2, that is, the droplet LD2 is on both the driving electrodes 1 and 2. Referring to FIG. 5E and FIG. 5F, the driving electrodes 1 and 1′ are powered off, and the driving electrodes 3, 3′, and 4 are powered on, that is, the driving electrodes 2, 2′, 3, 3′, 4, and 6b are in an open state, so that a portion of the droplet LD1′ is driven to move in a direction away from the additional driving electrode 2′, and the droplet LD1′ is split into droplets LD1 and LD3; the droplet LD3 is on the additional driving electrode 2′ and has the second volume (for example, 2 μL), and the droplet LD1 is on the driving electrode 6b and the additional driving electrode 3′ and has the third volume (for example, 5 μL); because the driving electrode 6b and the additional driving electrode 3′ are enough to accommodate the droplet LD1, although the driving electrode 4 is turned on, the droplet LD1 will not move onto the driving electrode 4; in addition, the droplet LD2 is driven by the driving electrodes 2/3/4 and moves onto the driving electrodes 2 and 3; similarly, because the driving electrodes 2 and 3 are enough to accommodate the droplet LD2, the droplet LD2 will not move onto the driving electrode 4, but the present disclosure is not limited to this. In this step, powering on the driving electrode 4 can increase the driving force for the droplet LD1′, thereby facilitating the splitting of the droplet LD1′.

In some embodiments, the driving method for generating additional droplets through the droplet generation module B-4′ may include, for example: after generating the droplet LD1 with the third volume (for example, 5 μL) through the steps of FIG. 5A to FIG. 5C, the droplet LD1 is split into a droplet with the second volume (for example, 2 μL) and an additional droplet, and the volume of the additional droplet is the third volume minus the second volume (for example, 3 μL). For example, after the droplet LD1 is generated in the step of FIG. 5C, the electrodes 3′ and 6b are powered off, and the electrodes 1′ and 2′ are powered on, thereby driving the droplet LD1 to move onto the electrodes 1′ and 2′; because the volume of the droplet LD1 is larger than the total accommodatable volume of the electrodes 1′ and 2′, a portion of the droplet LD1 is still on the electrode 6b; thereafter, the electrode 1′ is powered off and the electrode 6b is powered on, thereby splitting the droplet LD1 into a droplet with the second volume on the electrode 2′ and an additional droplet on the electrode 6b.

FIG. 4B illustrates a plan view of the droplet generation module B-4′ according to some other embodiments of the present disclosure. The relative positional relationship of the plurality of driving electrodes in the droplet generation module B-4′ is substantially the same as the relative positional relationship of the plurality of driving electrodes in the droplet generation module B-4 shown in FIG. 4A, and the difference is that the dimensions of the respective driving electrodes in the droplet generation module B-4′ are different from those in the droplet generation module B-4. For example, in some embodiments, the droplet generation module B-4′ is configured to generate the first droplet having the first volume and the second droplet having the second volume; in this embodiment, the volume of a droplet that can be accommodated and driven by each driving electrode (i.e., the additional driving electrode 1′, 2′) having the second body area is approximately equal to the first volume of the first droplet (e.g., 1 μL), and the volume of a droplet that can be accommodated and driven by a single driving electrode (i.e. the driving electrode 2, 3 or 4) or a combination of driving electrodes (i.e., a combination of the driving electrode 6b and two additional driving electrodes 3′) having the first body area is approximately equal to the second volume of the second droplet (e.g., 2 μL).

In this embodiment, the dimensions of the additional driving electrodes 1′ and 2′ are approximately the same as the dimensions of the square electrodes in the droplet generation module B-1 shown in FIG. 3A, the dimensions of the driving electrodes 2, 3, 4 are approximately the same as the dimensions of the square electrodes in the droplet generation module B-2 shown in FIG. 3B, and the dimensions of other driving electrodes are set accordingly. The driving method for generating the droplet with the first volume or the second volume using the droplet generation module B-4′ is substantially the same as that shown in FIG. 5A to FIG. 5F, and will not be described again.

FIG. 4C illustrates a schematic plan view of a droplet generation module B-4″ according to some other embodiments of the present disclosure.

Referring to FIG. 4C, in some embodiments, the droplet generation module B-4″ is configured to generate droplets of three volume types (e.g., the first volume, the second volume, the third volume). For example, the droplet generation module B-4″ adds a fourth additional driving electrode (for example, an electrode 4′) and a fifth additional driving electrode (for example, an electrode 5′) on the basis of the droplet generation module B-4. The fourth and fifth additional driving electrodes are arranged on a side of the second additional driving electrode away from the first additional driving electrode in the first sub-direction d1, and the fourth additional driving electrode and the fifth additional driving electrode have the same third body area as each other, the third body area is smaller than the second body area. The third body area may correspond to the minimum droplet volume that the droplet generation module B-4″ can generate. Furthermore, in this example, in order to improve the accuracy of droplet generation, the second additional driving electrode is split into an electrode 2′ and two additional electrodes 6′, and the sum of the areas of the electrodes 2′ and 6′ is approximately equal to the second body area; and other structures of the droplet generation module B-4″ are similar to those of the droplet generation module B-4, and will not be described again here. In some embodiments, in the droplet generation module B-4″, the volumes of droplets that can be accommodated and driven by driving electrodes (or a combination of driving electrodes) having the first body area, the second body area, and the third body area are respectively equal to the third volume, the second volume and the first volume. For example, the additional driving electrodes 4′ and 5′ are both square electrodes and have the same size as the square electrodes in the droplet generation module B-1. The additional driving electrode 2′ has a long edge extending along the first sub-direction d1 and a short edge extending along the second sub-direction d2, the length of the long edge is approximately equal to the side length of the electrode 1′, and the length of the short edge is approximately equal to the side length of the electrode 4′. Electrodes with similar numeral references are electrically connected to each other, for example, electrodes 2 and 2′ are electrically connected, electrodes 4 and 4′ are electrically connected, electrodes 5 and 5′ are electrically connected, and electrodes 6b and 6′ are electrically connected.

In some embodiments, the operation process of using the droplet generation module B-4″ to generate droplets with the second volume and the third volume is substantially the same as the operation process of using the droplet generation module B-4 in FIG. 5A to FIG. 5F, and will not be described again here.

In some embodiments, generating the droplet with the first volume (e.g., 1 μL) through the droplet generation module B-4″ may use the driving method as shown in FIG. 5G to FIG. 5K.

For example, referring to FIG. 5G, after completing the sample adding operation, using the same method shown in FIG. 5A and FIG. 5B to drive the droplet LD0 onto the electrodes 2, 3, 4, 6b, 3′; referring to FIG. 5G to FIG. 5H, then, the electrodes 3, 3′, and 4 are powered off, and the electrode 5 is powered on, that is, the electrodes 2, 5, and 6b are in an open state to split the droplet LD0 into the droplet LD1 and the droplet LD2; referring to FIG. 5H to FIG. 5I, the electrodes 5 and 6b are powered off, and the electrodes 1 and 1′ are powered on, thereby driving the droplet LD1 to move onto the electrodes 1′ and 2′, and driving the droplet LD2 to move onto the electrodes 1 and 2; referring to FIG. 5I and FIG. 5J, the electrodes 1 and 1′ are powered off, the electrodes 4, 4′, 5, and 5′ are powered on, thereby driving the droplet LD1 to move onto the electrodes 2′, 4′, and 5′; and referring to FIG. 5J and FIG. 5K, the electrode 4′ is powered off, and the electrode 6′ is powered on, thereby splitting the droplet LD1 into a droplet LD3 and a droplet LD4, and the droplet LD3 has the first volume.

It should be noted that in various embodiments of the present disclosure, the first sub-direction d1 described with respect to each droplet generation module is referred to as the arrangement direction of corresponding driving electrodes (for example, main electrodes 2, 3, 4, 6) in the droplet generation module; and correspondingly, the second sub-direction d2 is a direction perpendicular to the arrangement direction. In the microfluidic chip, the first sub-directions d1 of the plurality of droplet generation modules may be the same or different. For example, as shown in FIG. 1A, the first sub-directions d1 of the droplet generation modules B-2 and B-3 (that is, the arrangement direction of the corresponding electrodes) are the same, for example, both the same as the direction D1, whereas the first sub-direction d1 of the droplet generation module B-1 (that is, the arrangement direction of the corresponding electrodes) is different from (e.g., approximately perpendicular to) that of the droplet generation module B-2 or B-3. The sub-directions of respective droplet generation modules may be set and adjusted according to product design and requirements, which are not limited in the present disclosure.

Referring to FIG. 1A, in some embodiments, the droplet generation module B-1 is directly connected to the region D (including the first droplet driving module IM1); in the embodiments in which the digital microfluidic chip includes the droplet generation module B-4′ or B-4″, the droplet generation module B-4′ or B-4″ (for example, the additional driving electrode 2′ or 5′ at the end thereof) may also be directly connected to the first droplet driving module IM1; for example, at least a portion of the first droplet driving module IM1 may be reused as the droplet cutting module CM. In an alternative embodiment, the first droplet driving module IM1 may not be reused as the droplet cutting module CM, and the droplet cutting module CM may be separately provided in other regions.

In some embodiments, the first droplet driving module IM1 includes a plurality of driving electrodes, and the plurality of driving electrodes have substantially the same size and are arranged along the direction D1. One or more droplet generation modules B-1 are directly connected to the first droplet driving module IM1. In the case that there are a plurality of droplet generation modules B-1, the plurality of droplet generation modules B-1 may be arranged on opposite sides of the first droplet driving module IM1 in a direction D2 perpendicular to the direction D1, so that relatively more droplet generation modules B-1 may be arranged. The directions D1 and D2 may be referred to as a first direction and a second direction respectively. The driving electrode 6 at the end of the droplet generation module B-1 is directly connected to a corresponding electrode of the first droplet driving module IM1, and the arrangement direction of main electrodes in the droplet generation module B-1 (i.e., the first sub-direction d1) is substantially perpendicular to the arrangement direction of electrodes in the first droplet driving module IM1 (i.e., the first direction D1).

In some embodiments, the droplet receiving module RM is configured to receive a plurality of droplets generated by a plurality of droplet generation modules, and can implement operations such as mixing and reaction of the plurality of droplets. For example, the droplet receiving module RM is configured to at least implement mixing and a first reaction of liquid within a first mixing volume range, and implement mixing and a second reaction of liquid within a second mixing volume range, and the first mixing volume range is different from the second mixing volume range. In addition, a droplet splitting operation may also be performed on the droplet in the droplet receiving module RM. The droplet splitting performed in the droplet receiving module RM is, for example, to split a large-volume droplet into small droplets to achieve liquid transfer.

In some embodiments, the droplet receiving module RM includes a region C and a region A. The region C and the region A may be referred to as a first reaction region C and a second reaction region A, respectively. In some embodiments, the first reaction region C is configured to at least implement mixing and the first reaction of liquid within the first mixing volume range.

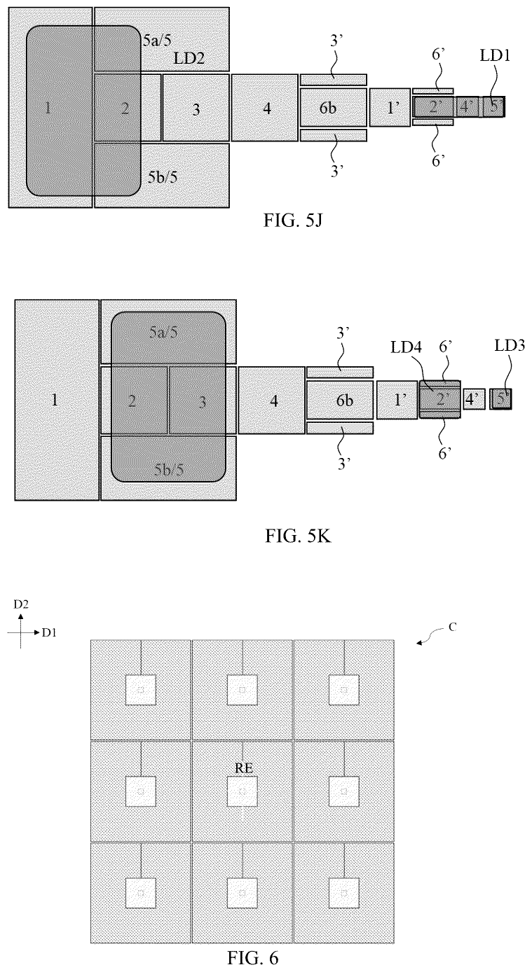

FIG. 6 shows an enlarged schematic diagram of a first reaction region C according to some embodiments of the present disclosure.

Referring to FIG. 1A and FIG. 6, in some embodiments, the first reaction region C includes a plurality of electrodes (or reaction electrodes) RE arranged in an array; for example, the plurality of reaction electrodes RE are arranged in a 3×3 array along directions D1 and D2, but the present disclosure is not limited thereto. The reaction electrode RE may also be referred to as a first reaction electrode.

In some embodiments, the volume of a droplet that can be accommodated and/or driven by each reaction electrode RE is greater than the first volume and the second volume and less than the third volume, and for example, the maximum value of the first mixing volume range of a mixed droplet that can be accommodated by the first reaction region C constituted by a plurality of reaction electrodes RE is an integer multiple of the third volume. The region E on a side of the reaction region C and directly connected to the reaction region C includes a plurality of driving electrodes arranged in the direction D1, and the volume of a droplet that can be accommodated and driven by each driving electrode may be the same as or different from the volume of a droplet that can be accommodated and driven by each reaction electrode; and in some embodiments, the volume of the droplet that can be accommodated and driven by each driving electrode may be greater than the volume of a droplet that can be accommodated and driven by each reaction electrode. For example, the volume of the droplet that can be accommodated and driven by each connection electrode may be approximately equal to the third volume. For example, the region E may be or include the droplet driving module IM3.

In some examples, the reaction region C is constituted by 3×3 reaction electrodes each with a side length of 1645 μm, and is configured to implement mixing, reaction, and other operations of droplets with a maximum mixing volume of 20 μL, and the manipulatable volume of the droplet that can be manipulated by a single reaction electrode RE is approximately 2.3 μL. However, the present disclosure is not limited thereto. It should be understood that the size and number of electrodes in the reaction region may be set and adjusted according to actual product design and requirements; for example, more or less reaction electrodes may be included in the reaction region, and the reaction electrodes may have other types of shapes and/or adopt other types of arrangements to implement mixing, reaction and other operations of liquid with larger or smaller mixing volume.

In some embodiments, the second reaction region A is configured to implement mixing and the second reaction of liquid within the second mixing volume range. The second reaction region may include a plurality of second reaction electrodes, and for example, the plurality of second reaction electrodes may be arranged in the direction D1. The volume of a droplet that can be accommodated and driven by each second reaction electrode is an integer multiple of the third volume, and may be larger than the volume of a droplet that can be accommodated and driven by a single electrode in region C or E, but the present disclosure is not limited thereto.

In some embodiments, when performing droplet reaction operation, a magnetic bead may be used for a purification operation, or part of the reaction needs to be performed within a suitable temperature range. In some embodiments, a magnetic control region may be included in the droplet receiving module (e.g., reaction region) to facilitate the manipulation of magnetic bead droplet. In some embodiments, the reaction region may include a temperature control region such that liquid in the temperature control region may react at a temperature within a predetermined temperature range. For example, the first reaction region C may include a magnetic control region, and the second reaction region A may include a temperature control region. In some examples, the second reaction region A may include a first temperature control region, a second temperature control region, and a third temperature control region, which are configured to control liquid in a corresponding region to react at a temperature within a first temperature range, a second temperature range, and a third temperature range, respectively; and the first temperature range, the second temperature range and the third temperature range are different from each other. For example, the second reaction region A including three temperature control regions may be used to perform a three-temperature polymerase chain reaction (PCR amplification reaction), but the present disclosure is not limited thereto. In alternative embodiments, the second reaction region may include two temperature control regions (for example, used for a two-step PCR amplification reaction), a single temperature control region (for example, used for a constant-temperature PCR amplification reaction), or more temperature control regions, according to actual application requirements.

In some embodiments, the interconnection module IM includes a plurality of droplet driving modules, thereby achieving interconnection between a plurality of droplet generation modules, droplet receiving modules, droplet cutting modules and/or other regions. For example, the interconnection module IM may include droplet driving modules IM1, IM2, and IM3; and the respective droplet driving modules in the interconnection module IM may be arranged along the direction D1 and/or the direction D2, and may be connected to each other. For example, the droplet driving modules IM1 and IM3 may be connected through the droplet driving module IM2. Among the droplet generation modules, the droplet generation module for generating the second droplet may be connected to the droplet driving module IM2, and the droplet generation module for generating the third droplet may be connected to the droplet driving module IM2 or IM3. The reaction regions A and C may each be connected to at least one of the droplet driving modules IM1, IM2, and IM3.

For example, the droplet generation modules B-2 and B-3 are both directly connected to the droplet driving module IM2, and the reaction region C is directly connected to the droplet driving modules IM2 and IM3; and for example, the droplet driving module IM3 may be arranged on opposite sides of the reaction region C in the direction D1. In some embodiments, the reaction region C is directly connected to a plurality of different types of droplet driving modules in the interconnection module IM, which can improve the flexibility of the droplet movement path and/or shorten the droplet movement path.

It should be understood that, except for the additional dummy electrodes in the droplet generation module, the electrode arrays in each module in the digital microfluidic chip 500 include the driving electrode structure DS as shown in FIG. 1C, and adjacent driving electrode structures are spaced apart by a certain distance. For the sake of simplicity of the drawings, FIG. 1A does not explicitly show the gap between adjacent electrode structures DS, but it should be understood that there is a gap between adjacent electrode structures such that the adjacent electrode structures are spaced apart from each other without contacting each other, so that the manipulation of droplets can be achieved reliably and effectively.

In some embodiments, as shown in FIG. 1A, among the plurality of modules in the digital microfluidic chip 500, the reaction regions A and C may be provided at two ends, and the droplet generation structure and the interconnection module are provided between the reaction regions A and C. This arrangement ensures that the droplet generation module is kept away from the temperature control region or magnetic control region. As such, droplets formed by the droplet generation modules do not pass through the temperature control region or magnetic control region before entering the reaction region for reaction, which avoids any adverse effects on the manipulation and/or properties of the droplets by the temperature control region or magnetic control region before the droplets reaction.

In some embodiments, the droplet cutting module CM is located at a relatively middle position, so that the distance between each droplet generation module and the droplet cutting module CM is relatively short, such that the movement path of the droplet to be cut is relatively short during the cutting process.

In some embodiments, other electrode array regions of the digital microfluidic chip 500 except the droplet generation modules and the reaction region C may select electrode arrays with appropriate dimensions according to requirements, and the dimensions of the driving electrodes in different modules may be different, and the dimensions of a plurality of driving electrodes in the same module may also be the same as or different from each other.

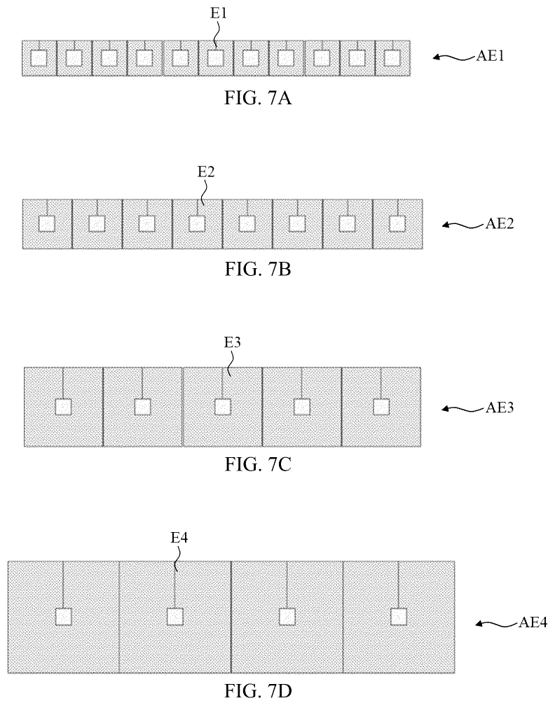

FIG. 7A to FIG. 7D are schematic enlarged plan views of various electrode arrays included in a digital microfluidic chip according to some embodiments of the present disclosure.

Referring to FIG. 7A to FIG. 7D, in some embodiments, the digital microfluidic chip 500 includes electrode arrays AE1, AE2, AE3, and AE4 in different regions. The electrode arrays AE1, AE2, AE3, and AE4 respectively include a plurality of electrodes E1, E2, E3, and E4 arranged in a row or a column; and in each electrode array, the plurality of electrodes are spaced apart from each other by a certain distance, and substantially aligned with each other in the electrode arrangement direction. It should be understood that, for ease of description, FIG. 7A to FIG. 7D show the plurality of electrodes of the electrode arrays AE1-AE4 as being arranged in the same direction, but in the chip, each electrode array may be arranged as needed in the direction D1 or D2 shown in FIG. 1A or any suitable direction.

In each of the electrode arrays AE1-AE4, the plurality of driving electrodes may have substantially the same shape and dimension as each other and be spaced apart from each other by a certain distance. In some embodiments, the electrodes E1-E4 may all be square electrodes, and the electrodes E1-E4 have dimensions different from each other. For example, the areas of the electrodes E1-E4 increase sequentially and may be designed into appropriate dimensions according to actual application requirements. That is, the manipulatable volumes of droplets that can be manipulated by the electrode arrays AE1-AE4 increases sequentially. In some embodiments, in each of the electrode arrays AE1-AE4, the minimum volume of a drivable droplet that can be driven by an electrode array is the volume of a droplet that can be accommodated and driven by a single electrode in the electrode array, the maximum volume of a drivable droplet that can be driven by an electrode array is the volume of a droplet that can be accommodated and driven by three electrodes in the electrode array.

For example, in the electrode array AE1, the volume of a droplet that can be driven by a single electrode E1 is approximately equal to the first volume, and the maximum volume of a droplet that can be driven by the electrode array AE1 is three times the first volume (that is, a corresponding droplet occupies three electrodes E1). Therefore, the volume range of the drivable droplet that can be driven by the electrode array AE1 is from the first volume (a corresponding droplet occupies one electrode E1) to three times the first volume (a corresponding droplet occupies 1-3 electrodes E1).

In the electrode array AE2, the volume of a drivable droplet that can be driven by a single electrode E2 is approximately equal to the second volume, and the maximum volume of a drivable droplet that can be driven by the electrode array AE2 is three times the second volume (that is, a corresponding droplet occupies three electrodes E2). Therefore, the volume range of the drivable droplet that can be driven by the electrode array AE2 is from the second volume to three times the second volume (a corresponding droplet occupies 1-3 electrodes E2).

In the electrode array AE3, the volume of a drivable droplet that can be driven by a single electrode E3 is approximately equal to the third volume, and the maximum volume of a drivable droplet that can be driven by the electrode array AE3 is three times the third volume (that is, a corresponding droplet occupies three electrodes E3). Therefore, the volume range of the drivable droplet that can be driven by the electrode array AE3 is from the third volume to three times the third volume (a corresponding droplet occupies 1-3 electrodes E3).

In the electrode array AE4, the volume of a drivable droplet that can be driven by a single electrode E4 is an integer multiple of (e.g., twice) the third volume, and the maximum volume of a drivable droplet that can be driven by the electrode array AE4 is six times the third volume (that is, a corresponding droplet occupies three electrodes E4). Therefore, the volume range of the drivable droplet that can be driven by the electrode array AE4 is from twice the third volume to six times the third volume (a corresponding droplet occupies 1-3 electrodes E4).

In some examples, the first volume, the second volume, and the third volume are 1 μL, 2 μL, and 5 μL, respectively, and the electrodes E1-E4 are all square electrodes; correspondingly, the side length of the electrode E1 is approximately 1084 μm, the spacing between adjacent electrodes E1 is approximately 25 μm, and the electrode array AE1 can realize the movement of a droplet in a volume range of 1-3 μL; the side length of the electrode E2 is approximately 1550 μm, the spacing between adjacent electrodes E2 is approximately 25 μm, and the electrode array AE2 can realize the movement of a droplet in a volume range of 2-6 μL; the side length of the electrode E3 is approximately 2474 μm, the spacing between adjacent electrodes E3 is approximately 25 am, and the electrode array AE3 can realize the movement of a droplet in a volume range of 5-15 L; and the side length of the electrode E4 is approximately 3500 am, the spacing between adjacent electrodes E4 is approximately 25 μm, and the electrode array AE4 can realize the movement of a droplet in a volume range of 10-30 μL. In some embodiments, for the relatively large electrode arrays AE3 and AE4, in order to improve the manipulation accuracy of the droplet, the droplet is allowed to occupy at most two electrodes in the electrode array AE3 or AE4 during the droplet manipulation, that is, the electrodes Array AE3 corresponds to the movement of a droplet of 5-10 μL, and electrode array AE4 corresponds to the movement of a droplet of 10-20 μL.

Referring to FIG. 1A and FIG. 7A to FIG. 7D, in some embodiments, in order to ensure that droplets generated by the plurality of droplet generation modules can be driven by the electrode array, the connection and arrangement between the droplet generation modules and the electrode arrays need to follow the following rules: in the droplet generation module and the electrode array connected to each other, the minimum volume of the droplet generated by the droplet generation module needs to be smaller than or equal to the maximum volume of the drivable droplet that can be driven by the electrode array, and larger than or equal to the minimum volume of the drivable droplet that can be driven by the electrode array. Therefore, the droplet generation module B-1 can be connected to the electrode array AE1, the droplet generation module B-2 can be connected to the electrode array AE1 or AE2, and the droplet generation module B-3 can be connected to the electrode array AE2 or AE3. After the above conditions are met, the positions of the droplet generation modules may be further set according to the characteristics of the biochemical reaction and chip layout.

In some embodiments, in order to ensure that the digital microfluidic chip can achieve manipulation of droplets with a wide range of volumes, the connection, arrangement, and movement of droplets between electrode arrays follow the following rules: (1) in the case where a droplet of a small-sized electrode array needs to be directly driven by a large-sized electrode array, the maximum volume of the drivable droplet that can be driven by the small-sized electrode array needs to be greater than the minimum volume of the drivable droplet that can be driven by the large-sized electrode array; (2) in the case where a droplet of a small-sized electrode array does not need to be directly driven by a large-sized electrode array, the droplet on the small-sized electrode array is driven by the electrode(s) in the large-sized electrode array and can move unidirectionally toward the large-sized electrode array, and only when the cumulative volume of the moving droplets is greater than the minimum volume of the drivable droplet that can be driven by the large-sized electrode array, the accumulated droplet can move between the electrodes in the large-sized electrode array; (3) in the case where a droplet of a large-sized electrode array needs to be directly driven by a small-sized electrode array, the droplet on the large-sized electrode array needs to be split into a droplet whose volume is within the volume range of the drivable droplet that can be driven by the small-sized electrode array, then the droplet can move between the electrodes of the small-sized electrode array. After the above conditions are met, the positions of electrode arrays may be further set according to actual application requirements (for example, the characteristics of biochemical reactions and chip layout, etc.).

It should be noted that here, the small-sized electrode array and the large-sized electrode array are described relative to two electrode arrays that are connected to each other; for example, the first electrode array and the second electrode array are connected to each other, and the size of a single electrode in the first electrode array is smaller than the size of a single electrode in the second electrode array, then the first electrode array is referred to as a small-sized electrode array, and the second electrode array is referred to as a large-sized electrode array.

Referring to FIG. 1A and FIG. 7A to FIG. 7D, in some embodiments, based on the above rules, in the digital microfluidic chip 500, the region D (i.e., the droplet driving module IM1) directly connected to the droplet generation module B-1 adopts an electrode array AE1, and the volume of a single droplet configured to be generated in the droplet generation module B-1 corresponds to (e.g., the same as) the minimum volume of a drivable droplet that can be driven by a single electrode in the electrode array AE1. In some embodiments, the droplet cutting module CM1 also includes an electrode array AE1, and the droplet driving module IM1 and the droplet cutting module CM1 may share the same electrode array AE1, but the present disclosure is not limited thereto. The droplet driving module IM2 in the interconnection module IM adopts an electrode array AE2. The droplet driving module IM2 can connect various modules and/or regions, and connect the droplet driving modules IM1 and IM3. The reaction region C adopts the electrode array shown in FIG. 6, and the region E (i.e., the droplet driving module IM3) on a side of the reaction region C adopts an electrode array AE3; and the reaction region A adopts an electrode array AE4. In some embodiments, the reaction region C is directly connected to both the electrode arrays AE2 and AE3 having different sizes (i.e., different driving capabilities), which can improve the flexibility of the droplet movement path and/or shorten the droplet movement path. In some other embodiments, the interconnection module IM may also include electrode arrays of other sizes according to actual application requirements.

In some embodiments, among the plurality of droplet driving modules of the interconnection module IM, the droplet driving module IM1 adopting the electrode array AE1 is mainly configured to connect the droplet generation module B-1; the droplet driving module IM3 adopting the electrode array AE3 is mainly configured to connect the reaction region C; and the droplet driving module IM2 adopting the electrode array AE2 is mainly configured for the connection with other modules and/or regions, and the connection with the droplet driving modules IM1 and IM3. In some embodiments, in the interconnection module IM, the number of droplet driving modules IM2 is greater than the number of droplet driving module(s) IM1 and the number of droplet driving module(s) IM3.

In some embodiments, in order to be compatible with as many biochemical processes as possible, as many droplet generation modules as possible may be provided in the digital microfluidic chip, and the droplet generation modules need to be as far away as possible from the temperature control or magnetic control region (for example, in the reaction region) to avoid the temperature control or magnetic control region from adversely affecting the droplets before reaction. In some embodiments, because the volume of a single droplet generated by the droplet generation module B-1 (i.e., the minimum volume of a droplet that the module can generate) is consistent with (e.g., substantially the same as) the minimum volume of the drivable droplet that can be driven in the region D, the droplet generation module B-1 needs to be directly connected to the region D to realize the movement of the droplet generated by the droplet generation module B-1. In order to maximize the number of droplet generation modules B-1 to improve the flexibility of droplet generation, that is, in order to provide as many droplet generation modules B-1 as possible, the droplet generation modules B-1 may be arranged on opposite sides of the region D in the direction D2, and may be evenly distributed. In some embodiments, because the interconnection module IM connecting respective regions mainly adopts the electrode array AE2, and the volume of a single droplet generated by the droplet generation module B-2 (i.e., the minimum volume of a droplet that the module can generate) is consistent with the minimum volume of the drivable droplet that can be driven by the electrode array AE2, more droplet generation modules B-2 may be provided. For example, in some embodiments, the number of droplet generation modules B-2 is greater than the number of droplet generation module(s) B-1 and/or is greater than the number of droplet generation module(s) B-3.

On the other hand, the plurality of droplet generation modules need to include at least droplet generation modules that can generate droplets with the first volume, the second volume, and the third volume; the plurality of droplet generation modules may include a generation module that can generate only a single volume type of droplet (for example, droplet generation modules B-1, B-2, B-3), a generation module that can generate multiple volume types of droplets (for example, droplet generation modules (or may be referred to as the fourth droplet generation module) B-4, B-4′, B-4″), or combinations thereof. For example, in the case where the droplet generation modules are all generation modules that can generate a single volume type of droplet, the droplet generation modules includes at least one droplet generation module B-1, at least one droplet generation module B-2 and at least one droplet generation module B-3; in some other examples, the droplet generation modules may include at least one first droplet generation module B-1 and at least one droplet generation module B-4; or the droplet generation modules may include at least one third droplet generation module B-3 and at least one droplet generation module B-4′; or the droplet generation modules may include at least one droplet generation module B-4″.

In the example shown in FIG. 1A, the digital microfluidic chip 500 includes two first droplet generation modules B-1, three second droplet generation modules B-2, and one third droplet generation module B-3. In some examples, the droplet cutting module CM is located between reaction regions A and C, and a portion (e.g., a portion at the left in FIG. 1A) of the interconnection module IM connects the reaction regions A and C. The droplet cutting module CM may be connected to a driving electrode in the middle of the portion of the interconnection module IM, and one of the plurality of second droplet generation modules B-2 and the droplet generation module B-3 are located on the same side of the droplet cutting module CM in the direction D1, so as to ensure the shortest path that the droplets need to move during various operation steps. In some embodiments, a plurality of droplet generation modules connected to a portion of the interconnection module extending in the same direction are located on the same side of the portion of the interconnection module; for example, as shown in FIG. 1A, the droplet generation modules B-2 and B-3 connected to the portion of the interconnection module IM extending in the direction D1 are both on the left side of the portion of the interconnection module IM, thus saving chip area.

In some embodiments, the settings of the first volume, the second volume, and the third volume may be set according to the droplet manipulation volume required in the actual application of the chip; for example, in some examples, the digital microfluidic chip is configured to manipulate droplets with a volume range of 1 μL-20 μL and a step size of 1 μL, and for example, may be applied in the preparation of gene libraries. Based on this application requirement, generally in library preparation, the required volume of various reagents is generally less than 5 μL, and the volumes of 1-5 μL are all involved. In order to obtain droplets in a volume range of 1 L-20 μL and a step size of 1 μL, the droplet operation unit of 1 μL is necessary, so the first volume is set to 1 μL; considering the driving ability of the driving electrode for droplets, the volume of the next-level droplet operation unit (i.e., the second volume) needs to be less than 3 μL. If an operation unit of 3 μL is adopted, then droplets of 2 μL and 1 μL cannot be driven by an electrode array with a single electrode whose manipulable droplet volume is 3 μL; and if an operation unit of 2 μL is adopted, then a droplet of 1 μL cannot be driven by an electrode array with a single electrode whose manipulable droplet volume is 2 μL. Considering the connections between respective regions, if an electrode array with a single electrode manipulable droplet volume of 1 μL is adopted as the connection electrode array between the respective regions, the drivable droplet volume that can be driven by the connection electrode array is only 1-3 μL, and a relatively large number of electrodes need to be used, which will lead to a reduction in droplet driving efficiency and an increase in the number of bonding electrodes. Therefore, in order to improve the driving ability and driving efficiency of the connection electrode array for droplets, a droplet volume of 2 μL is selected as the volume of the next-level droplet operation unit, that is, the second volume is set to be 2 μL. In addition, in order to reduce the droplet operation steps, the volume of the next-level operation unit (i.e., the third volume) is set to be 5 μL.

In the case where the first volume, the second volume, and the third volume are set to be 1 μL, 2 μL, and 5 μL respectively, adopting the above electrode arrangement allows the acquisition and manipulation of droplets with a volume range of 1 μL-20 μL and a step size of 1 μL in the digital microfluidic chip 500. For example, in the example shown in FIG. 1A, a 1 μL droplet may be generated by the droplet generation module B-1, a 2 μL droplet may be generated by the droplet generation module B-2, and a 5 μL droplet may be generated by the droplet generation module B-3. In addition, a 4 μL droplet may be obtained by splitting a 1 μL droplet from a 5 μL droplet, and a 3 μL droplet may be obtained by splitting a 2 μL droplet (i.e., two 1 μL droplets) from a 5 μL droplet. The above-mentioned droplet splitting operations (or called cutting operations) may each be realized through the droplet cutting module CM. Alternatively, the 4 μL droplet may also be obtained by generating two 2 μL droplets in the droplet generation module B-2 and mixing the two droplets. A 6 μL droplet may be obtained by mixing a 5 μL droplet with a 1 μL droplet, a 7 μL droplet may be obtained by mixing a 5 μL droplet with a 2 μL droplet, an 8 μL droplet may be obtained by mixing a 5 μL droplet with a 3 μL droplet, a 9 μL droplet may be obtained by mixing a 5 μL droplet with a 4 μL droplet, a 10 μL droplet may be obtained by generating two 5 μL droplets in the droplet generation module B-3, and mixing the two droplets, and droplets with a volume above 10 uL may be obtained by combining droplets of different volumes.

In some embodiments, the method of generating multiple volume types of droplets using the digital microfluidic chip may include at least one of the following: generating one or more first droplets each having the first volume using a droplet generation module (e.g., droplet generation module B-1, B-4′ or B-4″); generating one or more second droplets each having the second volume using a droplet generation module (e.g., droplet generation module B-2, B-4, B-4′ or B-4″); generating one or more third droplets each having the third volume using a droplet generation module (e.g., droplet generation module B-3, B-4, B-4′, B-4″); generating at least one of one or more second droplets each having the second volume and one or more third droplets each having the third volume using a droplet generation module (e.g., droplet generation module B-4, B-4′, B-4″); generating at least one of one or more fourth droplets each having the fourth volume and one or more fifth droplets each having the fifth volume using a droplet generation module (e.g., droplet generation module B-3, B-4, B-4′, B-4″) and a droplet cutting module in combination, and the fourth volume and the fifth volume are greater than the second volume and less than the third volume; and mixing at least two of the one or more first droplets, the one or more second droplets, the one or more third droplets, the one or more fourth droplets, and the one or more fifth droplets to obtain droplets with other target volumes. In some examples, the above-mentioned first volume, second volume, third volume, fourth volume, and fifth volume are 1 μL, 2 μL, 5 μL, 3 μL, and 4 μL, respectively.

In some embodiments, generating the fourth droplet using the third droplet generation module and the droplet cutting module in combination, includes: generating an initial droplet with the third volume using the third droplet generation module; and cutting out two cut droplets each having the first volume from the initial droplet by the droplet cutting module, and moving the two cut droplets to the waste liquid region, where a remaining droplet of the initial droplet after being cut forms the fourth droplet.

In some embodiments, generating the fifth droplet using the third droplet generation module and the droplet cutting module in combination, includes: generating an initial droplet with the third volume using the third droplet generation module; and cutting out one cut droplet having the first volume from the initial droplet by the droplet cutting module, and moving the cut droplet to the waste liquid region, where a remaining droplet of the initial droplet after being cut forms the fifth droplet.

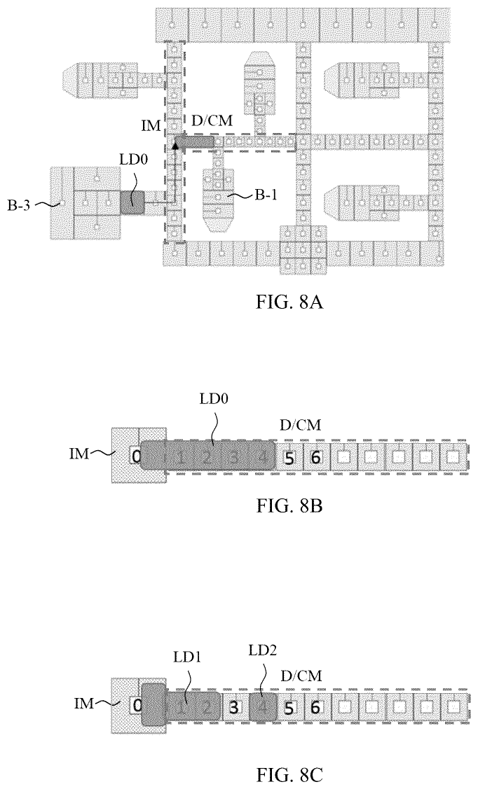

FIG. 8A to FIG. 8C show an example of generating a droplet having the target volume using the digital microfluidic chip shown in FIG. 1A through the droplet generation module B-3 and the droplet cutting module CM.

Referring to FIG. 8A, in some embodiments, a droplet LD0 having the third volume is generated by the droplet generation module B-3, by sequentially applying a driving signal to (i.e., powering on) the corresponding driving electrodes in the interconnection module IM connected to the droplet generation module B-3, the droplet LD0 is moved to the region D (i.e., the droplet cutting module CM) through the interconnection module IM. For example, the droplet cutting module CM includes driving electrodes 1, 2, 3, 4, 5 . . . arranged in the direction D1; the electrode 0 in the interconnection module IM is directly connected to the droplet cutting module CM, for example, directly connected to the electrode 1. The moving path of the droplet LD0 is shown in FIG. 8A, and FIG. 8B shows an enlarged schematic diagram of the droplet LD0 moving to the region D. In some embodiments, when the droplet LD0 moves to a position close to the droplet cutting module CM, the electrodes 1-4 are turned on, so that the droplet LD0 moves onto the electrodes 1-4; in some examples, because the volume of the droplet LD0 (that is, the third volume) is larger than the volume of the droplet that can be accommodated by the electrodes 1-4 (that is, four times the first volume), a portion of the droplet LD0 is still on the electrode 0, and the state of the droplet LD0 in this step is shown in FIG. 8B; referring to FIG. 8B and FIG. 8C, then, turning off the electrode 3 and turning on the electrode 0, so that the droplet LD0 is split into a droplet LD1 and a droplet LD2; thereafter, the droplet LD2 is driven to move to the waste liquid region, thereby obtaining the droplet LD1 having the target volume. In this example, the droplet generation module B-1 may be reused as the waste liquid region, and the discarded droplet LD2 may be removed to this module. That is, through the operations of FIG. 8A to FIG. 8C, the droplet LD2 having the first volume is cut out from the droplet LD0 having the third volume, thereby obtaining the droplet LD1 having the target volume (i.e., the third volume minus the first volume). In some examples, the first volume is 1 μL and the third volume is 5 μL, that is, a droplet with a target volume of 4 μL may be obtained through the above steps. In some examples, by repeating the above operations, a discarded droplet of the first volume may be further cut out from the droplet LD1, thereby obtaining a droplet with a target volume of, for example, 3 μL.

In the above embodiment, the plurality of electrodes in the droplet cutting module CM have the same size and are arranged in a single direction, but the present disclosure is not limited thereto.

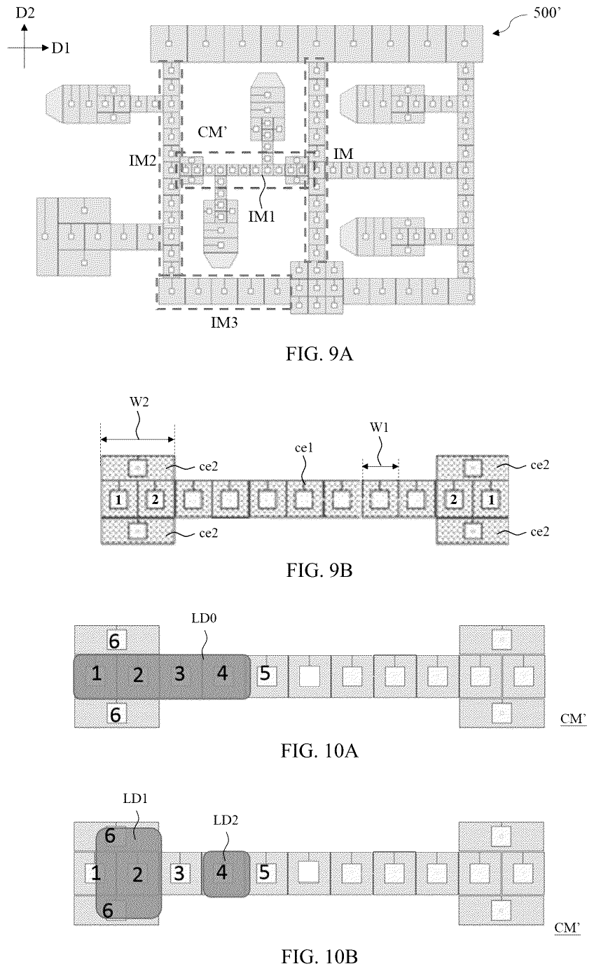

FIG. 9A illustrates a plan view of respective modules in a digital microfluidic chip 500′ according to some other embodiments of the present disclosure, and the digital microfluidic chip 500′ includes a droplet cutting module CM′; FIG. 9B illustrates an enlarged plan view of the droplet cutting module CM′ in FIG. 9A. The structure of the droplet cutting module CM′ is similar to the structure of the droplet cutting module CM in the previous embodiment, and the difference lies in that: the droplet cutting module CM′ further includes an auxiliary cutting electrode. Other structures of the digital microfluidic chip 500′ are similar to those of the digital microfluidic chip 500, and will not be described again here.

Referring to FIG. 9A and FIG. 9B, in some embodiments, the droplet cutting module CM′ includes a plurality of electrodes ce1 and one or more auxiliary electrodes ce2; and the electrode ce1 may also be called a main electrode or a main cutting electrode. The auxiliary electrode ce2 may also be called an auxiliary cutting electrode. Similar to the previous embodiments, the plurality of main electrodes ce1 have the same dimension (e.g., width, area, etc.) as each other, and for example, are arranged in the direction D1. One or more auxiliary cutting electrodes are arranged on one or opposite two sides of the main electrode array in a direction (e.g., direction D2) perpendicular to the arrangement direction (e.g., direction D1) of the main electrodes ce1. For example, the auxiliary cutting electrode may be connected to a cutting driving electrode ce1 close to the interconnection module, and may be connected to the interconnection module IM. The size of the auxiliary cutting electrode ce2 may be the same as or different from the size of the main electrode ce1. In this embodiment, at least part of the droplet cutting module CM′ (e.g., the main electrode) may share the same electrode array with the droplet driving module IM1.

In some embodiments, the main electrode ce1 has a width w1, the auxiliary cutting electrode ce2 has a width w2, and the width w2 is greater than the width w1, for example, the width w2 is twice the width w1; and the widths w1 and w2 are widths along the arrangement direction (for example, direction D1) of the main electrodes. For example, the main electrode ce1 is a square electrode with a side length w1, and the auxiliary cutting electrode ce2 is, for example, a rectangular electrode, with its long edge extending in the arrangement direction (for example, direction D1) of the main electrodes, and its short edge extending in the direction (for example, direction D2) perpendicular to the arrangement direction of the main electrodes. For example, the short edge and the long edge of the auxiliary cutting electrode ce2 may be approximately equal to one time and twice the side length of the main electrode ce1, respectively.

For example, among the plurality of main electrodes ce1, the electrodes 1 at both ends of the droplet cutting module CM′ in the direction D1 are directly connected to the driving electrodes of the interconnection modules (for example, the driving electrodes of the droplet driving modules IM2), respectively, and the electrode 2 is located on a side of the first electrode 1 away from the interconnection module IM and is directly connected to the electrode 1. One or more main electrodes at end(s) of the droplet cutting module CM′ may be referred to as end cutting electrodes or end main cutting electrodes. In some embodiments, the auxiliary cutting electrodes are located on opposite two sides of the end main cutting electrodes (e.g., electrodes 1 and 2) in direction D2, and the auxiliary cutting electrodes ce2 may be provided on each of the ends (e.g., two ends) of the droplet cutting module CM′ connected to the interconnection module, that is, the droplet cutting module CM′ may include four auxiliary cutting electrodes ce2. The auxiliary cutting electrode ce2 is immediately adjacent to and directly connected to the end main cutting electrode and the interconnection module. In some embodiments, the accuracy of droplet cutting may be improved by providing the auxiliary cutting electrode. It should be understood that the shape, dimension and number of the auxiliary cutting electrodes ce2 shown in FIG. 9A and FIG. 9B are only for illustration, and the present disclosure is not limited thereto.

FIG. 10A and FIG. 10B illustrate plan views of droplet cutting using the droplet cutting module CM′ shown in FIG. 9A and FIG. 9B according to some embodiments of the present disclosure.

Referring to FIG. 10A, the droplet LD0 is moved to the droplet cutting module CM′, for example, the electrodes 1-4 are turned on to move the droplet LD0 onto the electrodes 1-4; and the droplet LD0 may be a droplet from the droplet generation module B-3, a cut droplet, or a mixed droplet. If the droplet LD0 comes from the droplet generation module B-3, then a portion of the droplet LD0 is also located on an electrode, connected to the electrode 1, in the interconnection module, which is not shown here for the sake of simplicity of the drawings. After moving the droplet LD0 to the electrodes 1-4, turning off the electrode 3 and turning on the electrode 6, that is, the electrodes 1, 2, 4, and 6 are in an open state, so that the droplet LD0 is driven by the electrodes 1, 2, and 6, as well as by the electrode 4, to split into a droplet LD1 and a droplet LD2. Then, the droplet LD2 is moved to the waste liquid region, and the droplet LD1 is a droplet with the target volume.

In this embodiment, during the operation of droplet splitting, compared with the aforementioned embodiments of FIG. 8B and FIG. 8C that use electrodes 0, 1, and 2 to apply driving force, this embodiment uses electrodes 1, 2, and 6 to apply driving force to the droplets. The electrode 6 is closer to the droplet, which can better drive the splitting of the droplet and improve the accuracy of droplet cutting, that is, the accuracy of the volume of the obtained droplet can be improved; in the present disclosure, the greater the accuracy of the volume of the obtained droplet, the less the difference between the droplet volume and the target volume. In this embodiment, the structure of the droplet cutting module is similar to the structure of the droplet generation module, and the accuracy of the volume of the droplet obtained by cutting through the droplet cutting module is similar to the accuracy of the volume of the droplet generated by the droplet generation module.

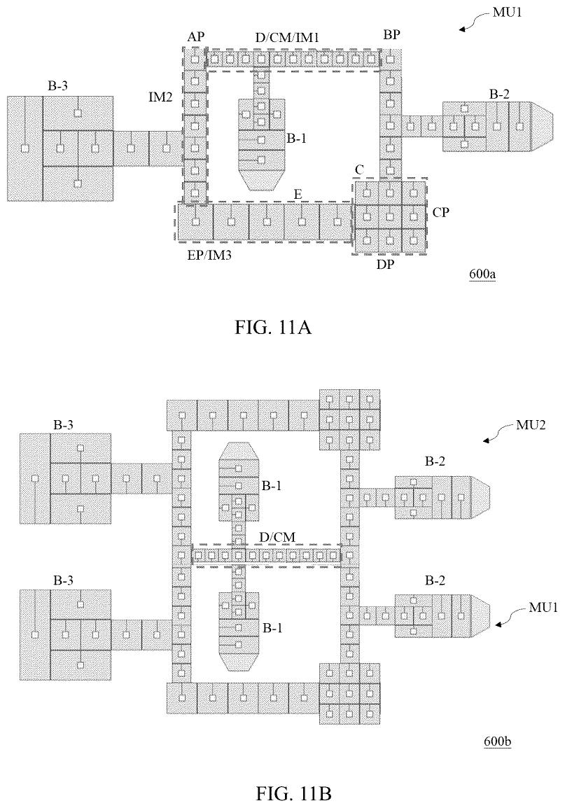

FIG. 11A and FIG. 11B show schematic plan views of digital microfluidic chips according to some other embodiments of the present disclosure. For the sake of simplicity of the drawings, FIG. 11A and FIG. 11B only show respective droplet manipulation modules in the digital microfluidic chip, without specifically showing other components. It should be understood that the other components are similar to those of the previous embodiments, and reference may be made to the structure shown in FIG. 1A.

Referring to FIG. 11A, respective driving modules in the digital microfluidic chip 600a are similar to those of the previous embodiment, and only the differences between this embodiment and the previous embodiment will be described below. In the digital microfluidic chip 600a, the droplet generation structure may include one droplet generation module B-1, one droplet generation module B-2, one droplet generation module B-3, and a droplet cutting module CM. The interconnection module IM includes droplet driving modules IM1, IM2, and IM3. For example, the region D directly connected to the droplet generation module B-1 is the droplet driving module IM1, and may be reused as the droplet cutting module CM. The droplet receiving module includes a reaction region C. The region E on a side of the reaction region C may be or include the droplet driving module IM3, and the droplet driving module IM3 is directly connected to the reaction region C. The respective modules are connected to each other through the interconnection module IM, and the arrangement rules between the respective modules are the same as those described above. In this embodiment, the reaction region C may be served as the first reaction region and has substantially the same function as the reaction region C in the previous embodiment; and the region E (i.e., the droplet driving module IM3) may be reused as the second reaction region, to achieve the same function as the reaction region A in the previous embodiment. That is, the droplet driving module IM3 and the second reaction region may share the same electrode array. For example, the region E may include a temperature control region for performing reactions that require temperature control, such as PCR amplification. In this example, the reaction region C may be configured to implement mixing and first reaction of liquid within the first mixing volume range, while the region E may be configured to implement mixing and second reaction of liquid within the second mixing volume range. In some other embodiments, the droplet receiving module includes a single reaction region C, and the reaction region C may be used to perform multiple types of reactions. For example, the reaction region C may include a magnetic control region, a temperature control region, etc. In this example, the reaction region C may be served as a common reaction region and be configured to implement mixing and the first reaction of liquid within the first mixing volume range, and implement mixing and the second reaction of liquid within the second mixing volume range in different operation steps.

In some embodiments, the module unit MU1 shown in FIG. 11A may be served as the smallest module unit (for example, the smallest reaction unit) of the microfluidic chip, and module units may be combined and/or expanded according to application requirements such as actual biochemical processes, to achieve compatibility with more biochemical processes and other application requirements. For example, in the module units of FIG. 11A, a plurality of expansion connection points AP, BP, CP, DP, EP may be included; here, “expansion connection point” refers to a location point where other electrode arrays and/or other modules may be further connected, allowing for the further expansion of the module unit.

For example, referring to FIG. 7A to FIG. 7D, and FIG. 11A, the expansion connection points AP and BP may be further connected to electrode arrays AE1, AE2 or AE3; the expansion connection points CP and DP may be further connected to electrode arrays AE1, AE2 or AE3; and the expansion connection point EP may be further connected to an electrode array AE2. The module units may be expanded according to actual application requirements.

FIG. 11B shows a schematic plan view of a droplet manipulation module in a digital microfluidic chip 600b according to some other embodiments of the present disclosure, and this module is an expanded module based on the module unit shown in FIG. 11A.

Referring to FIG. 11B, the droplet manipulation module of the digital microfluidic chip 600b includes a module unit MU1 and a module unit MU2 connected to each other. The module unit MU2 has a structure similar to that of the module unit MU1, and the two module units may share the region D as the droplet cutting module CM.

It should be understood that FIG. 11B is only an example of module unit expansion, and the present disclosure is not limited thereto. Various combinations and expansions of modules may be made according to actual application requirements.

In some embodiments of the present disclosure, a method of droplet mixing and/or reaction using a digital microfluidic chip is provided. The method may include: generating a sample droplet and/or various desired droplets through a droplet generation structure (e.g., one or more droplet generation modules and/or a droplet cutting module); driving the droplets to move to a droplet receiving module (e.g., a reaction region) by the driving electrodes of the interconnection module; and enabling the droplets to mix and/or react in the droplet receiving module. In some embodiments, a plurality of steps of mixing and/or reaction are required to be performed, and different mixing and reaction steps may be performed, for example, in different regions of the droplet receiving module, and the method may include driving the mixed droplet and/or the reacted droplet to move from one reaction region to another reaction region for the next step of reaction. This disclosure does not limit the application fields and reaction types of digital microfluidic chips.

In some embodiments of the present disclosure, a method for preparing a gene library using a digital microfluidic chip is provided. The method may include: adding a deoxyribonucleotide sample solution, a ligase buffer solution, a linker solution, and a ligase solution to corresponding droplet generation modules among the plurality of droplet generation modules, respectively, and generating a sample droplet, a ligase buffer droplet, a linker droplet, and a ligase droplet through the droplet generation structure; moving the sample droplet, the ligase buffer droplet, the linker droplet, and the ligase droplet to the droplet receiving module through the interconnection module, and the sample droplet, the ligase buffer droplet, the linker droplet, and the ligase droplet are mixed and reacted in a first reaction region of the droplet receiving module to form a sample mixture droplet; performing deoxyribonucleotide adsorption and elution on the sample mixture droplet to obtain a first deoxyribonucleotide chain; adding an amplification solution and a primer solution to corresponding droplet generation modules among the plurality of droplet generation modules, respectively, and generating an amplification droplet and a primer droplet through the droplet generation structure; moving the amplification droplet and the primer droplet to the droplet receiving module through the interconnection module, and the amplification droplet and the primer droplet with the first deoxyribonucleotide chain undergo a polymerase chain reaction in the droplet receiving module to form an amplified deoxyribonucleotide mixture droplet; and performing deoxyribonucleotide adsorption and elution on the amplified deoxyribonucleotide mixture droplet to obtain an amplified deoxyribonucleotide chain.

In some embodiments, the sample droplet has a sixth volume, and generating the sample droplet includes: adding the deoxyribonucleotide sample solution to a third droplet generation module, and generating a first sample droplet and a second sample droplet each having the third volume through the third droplet generation module; moving the second sample droplet to a droplet cutting module through the interconnection module; and cutting the second sample droplet into a cut sample droplet having the second volume and a third sample droplet having a remaining volume by the droplet cutting module, in which the first sample droplet and the third sample droplet constitute the sample droplet having the sixth volume.

In some embodiments, generating the ligase buffer droplet, the linker droplet, and the ligase droplet includes: adding the ligase buffer solution to a second droplet generation module, and generating the ligase buffer droplet having the second volume through the second droplet generation module; adding the ligase solution to the second droplet generation module, and generating two ligase sub-droplets each having the second volume through the second droplet generation module, in which the two ligase sub-droplets constitute the ligase droplet; and adding the linker solution to a first droplet generation module, and generating the linker droplet having the first volume through the first droplet generation module.

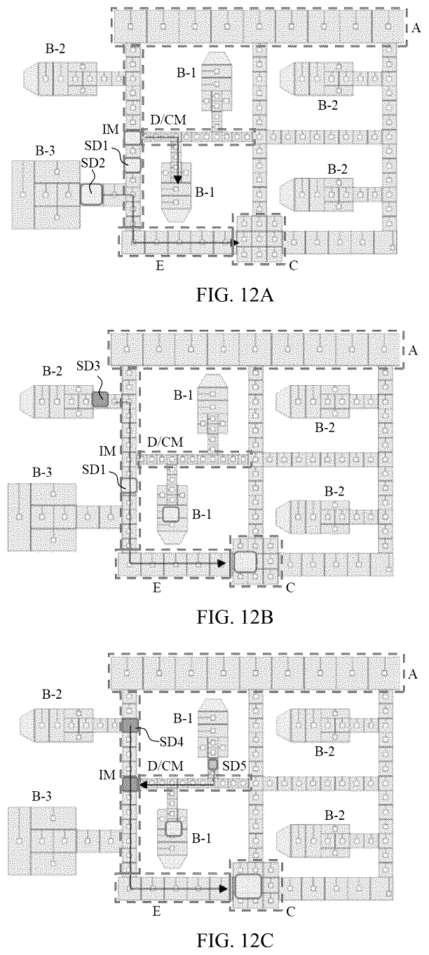

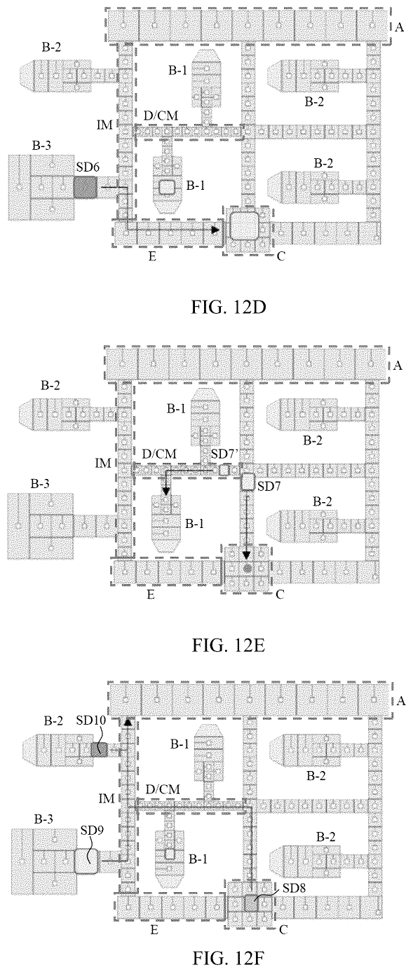

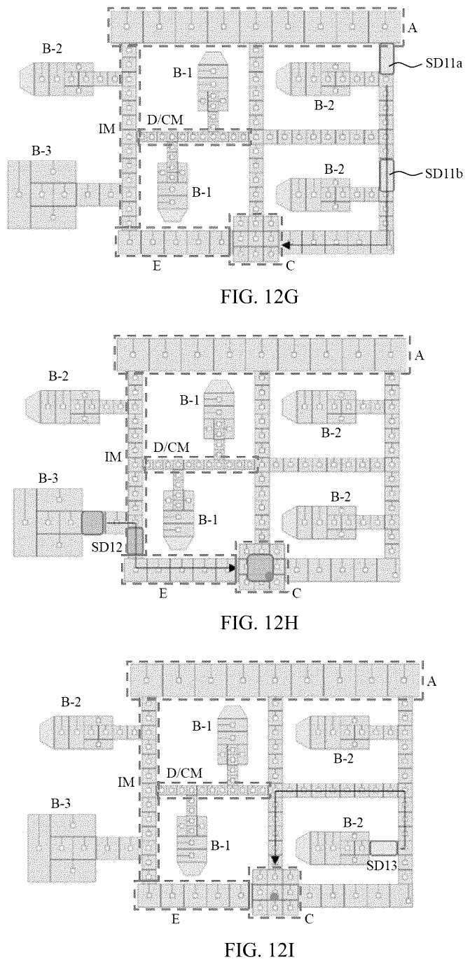

FIG. 12A to FIG. 12I show a method for preparing a gene library using a digital microfluidic chip 500 according to some embodiments of the present disclosure. The following takes the droplet generation modules B-1, B-2, and B-3 respectively configured to generate 1 μL, 2 μL, and 5 μL droplets as an example for explanation. The movement path of the droplets in each step is shown by the arrows in the corresponding figures.

Referring to FIG. 12A, a deoxyribonucleotide (DNA) sample droplet is obtained through a droplet generation structure (for example, one or more droplet generation modules and/or droplet cutting modules), for example, an 8 μL DNA sample droplet is obtained; and in order to minimize operational steps and reduce the driving distance of droplets, this step uses the droplet generation module B-3, the droplet generation module B-1 on a side (that is, at the lower side of the central region in the diagram) of the region D close to the droplet generation module B-3, the region D (droplet cutting module CM), the reaction region C in the droplet receiving module, and the droplet driving module IM3 on a side of the reaction region C, where the droplet generation module B-1 is served as a waste liquid pool in this step. For example, the DNA sample solution is injected through the sample inlet corresponding to the droplet generation module B-3, and a 5 uL DNA sample droplet is generated by the droplet generation module B-3; the 5 uL sample droplet is moved to the droplet cutting module CM by the driving electrodes of the interconnection module IM (for example, the droplet driving module IM2), a 2 uL waste droplet is cut out from the 5 uL sample droplet by the droplet cutting module CM and the waste droplet is moved to the waste liquid pool, thereby obtaining a 3 uL DNA sample droplet SD1; the 3 μL DNA sample droplet SD1 is moved to the electrode array at the left side of the chip; then, a 5 uL DNA sample droplet SD2 is regenerated by the droplet generator module B-3 again, and the sample droplet SD2 is moved into the reaction region C by the electrodes of the droplet driving modules IM2 and IM3 in the interconnection module IM.

Referring to FIG. 12B, the ligase buffer droplet is generated by the droplet generation structure, and the ligase buffer droplet is driven to move to the droplet receiving module by the driving electrodes in the interconnection module, and the ligase buffer droplet and the DNA sample droplet are mixed in the droplet receiving module (for example, the reaction region). In some embodiments, in order to minimize operational steps and reduce the driving distance of droplets to the greatest extent, this step uses the droplet generation module B-2 at the left and the region D (i.e., the droplet cutting module) to generate the ligase buffer droplet. For example, a ligase buffer solution is added from a sample inlet corresponding to the droplet generation module B-2, and a 2 uL ligase buffer droplet SD3 is generated by the droplet generation module B-2; the ligase buffer droplet SD3 is driven by the electrodes in the interconnection module IM to move to the location where the DNA sample droplet SD1 (the 3 uL droplet generated in the previous step) is located, such that the 2 uL ligase buffer droplet SD3 is mixed with the 3 uL DNA sample droplet SD1, and the mixed droplet is then driven to move to the reaction region C of the droplet receiving module, the movement path of the droplet is indicated by the arrow in FIG. 12B. In this example, the 3 μL DNA sample droplet SD1 was not moved to the reaction region in the previous step, but is moved to the reaction region C after being mixed with the ligase buffer droplet SD3 in this step. This is to make the mixed droplet be capable of being moved to the reaction region C through the droplet driving module IM3, which can shorten the movement path of the droplet, reduce the number of electrodes that the droplet crosses, and improve efficiency. In some other embodiments, the 3 μL DNA sample droplet SD1 may also be moved to the reaction region C in the previous step. Thus, in this step, the 2 uL ligase buffer droplet SD3 need to move through the region D and the interconnection module between the region D and the reaction region C to reach the reaction region C. In practical applications, the movement path of the droplet may be flexibly selected according to requirements.

Referring to FIG. 12C, a linker droplet and a ligase mix droplet are generated by the droplet generation module, and these droplets are mixed and then moved to the droplet receiving module (e.g., reaction region C). In some examples, in order to reduce the waste of reagents, this step uses the droplet generation module B-2 at the left and the droplet generation module B-1 on a side (i.e., the upper side of the middle region in the figure) of the region D close to the reaction region A to generate droplets. For example, after removing the liquid (i.e., the liquid remaining in the previous step) from the droplet generation module B-2 at the left, and a ligase mixture solution is added, and two 2 μL (i.e., 4 μL) ligase mixed droplets SD4 are generated by the droplet generation module B-2; a linker solution is added at the droplet generation module B-1, and a 1 uL linker droplet SD5 is generated by the droplet generation module B-1; then the three droplets (i.e., two 2 uL ligase mixed droplets SD4 and one 1 uL linker droplet SD5) are mixed and moved to the region C, where they are incubated at room temperature for 20 minutes. The movement path of the droplets is shown by the arrows in FIG. 12C. In some other embodiments, the 4 μL ligase droplet may also be obtained by generating a 5 μL droplet by the droplet generation module B-3, and cutting out 1 μL droplet from the 5 μL droplet, but using the droplet generation module B-2 to generate two 2 μL droplets to obtain the 4 μL ligase droplet avoids waste of reagents.

Referring to FIG. 12D, a magnetic bead droplet is obtained by a droplet generation structure, the magnetic bead droplet is driven to move to the droplet receiving module, and then a purification operation is performed, for example, in the reaction region C of the droplet receiving module, so that DNA is adsorbed onto the magnetic bead, then the liquid is discarded, and the magnetic bead with adsorbed DNA is retained. For example, residual liquid is removed from the droplet generation module B-3; then a magnetic bead (or purified magnetic bead) is added to the droplet generation module B-3, and a 5 uL droplet is generated; the magnetic bead droplet is then driven to the region C to be mixed with the droplet that was moved to the reaction region C in a previous step for purification operation, resulting in the DNA being adsorbed onto the magnetic bead. Then the liquid in the reaction region C is driven to the waste liquid region, with the magnetic bead carrying the DNA chain being retained.

Referring to FIG. 12E, an elution buffer droplet is obtained by the droplet generation structure, and the elution buffer droplet is driven to move to the droplet receiving module (for example, the reaction region C) by the interconnection module, so that the elution buffer droplet is mixed with the magnetic bead carrying the DNA chain and the elution of the DNA is completed, that is, the DNA chain is separated from the magnetic bead. For example, this step requires obtaining a 3 μL elution buffer droplet, and in order to reduce the number of sample adding modules used, the droplet generation module B-2 at the bottom right corner of the figure and the droplet cutting module CM may be selected to obtain the elution buffer droplet. An elution buffer solution is added to the droplet generation module B-2, and two 2 uL (i.e., a total of 4 uL) elution buffer droplets are generated by the droplet generation module B-2. These elution buffer droplets are then driven to move to the droplet cutting module CM where a 1 uL waste droplet SD7′ is cut out from the droplets, so as to obtain a 3 uL elution buffer droplet SD7. Then the elution buffer droplet SD7 is moved to the region C to complete the elution of DNA, and a DNA droplet SD8 is obtained, as shown in FIG. 12F.

Referring to FIG. 12F, an amplification droplet and a primer droplet are generated by the droplet generation structure, and the amplification droplet, the primer droplet, and the purified DNA droplet obtained in the previous step are driven to move to the reaction region (e.g., the reaction region A) in the droplet receiving module to perform the PCR amplification reaction. The residual liquid in the droplet generation module B-3 is removed, an amplification mix solution is added, and a 5 uL amplification droplet SD9 is then generated by the droplet generation module B-3. The residual liquid in the droplet generation module B-2 at the left side is removed, a primer solution is added, and a 2 uL primer droplet SD10 is generated by the droplet generation module B-2. The DNA droplet SD8, the amplification droplet SD9, and the primer droplet SD10 are then driven to the reaction region A. Then PCR amplification reaction is performed in the reaction region A. In some examples, a three-step PCR amplification reaction is used, and the reaction region includes three temperature control regions. For example, in the first temperature control region, the reaction takes place at 98° C. for 45 seconds, followed by 30 cycles of reaction. The cycle reaction conditions are reacting for 15 seconds at 98° C., then reacting for 30 seconds at 60° C., and finally reacting for 30 seconds at 72° C.

Referring to FIG. 12G, the liquid after completing the amplification reaction in the reaction region A is moved to the reaction region C. For example, the liquid in the reaction region A is split into two droplets SD11a and SD11b, and the two droplets are driven to move to the reaction region C by the driving electrodes of the interconnection module.

Referring to FIG. 12H, after the amplification reaction is completed, the magnetic bead droplet is obtained by the droplet generation structure, and the magnetic bead droplet is driven to move to the reaction region C of the droplet receiving module to perform purification operation on the amplified DNA solution; then the liquid is discarded, and the magnetic bead adsorbed with amplified DNA is retained. For example, the residual liquid is removed from the droplet generation module B-3, a magnetic bead is added, and two 5 uL purification droplets SD12 are generated. The magnetic bead and the purification droplets SD12 are moved to the reaction region C, where they are mixed with the droplets SD11a and SD11b which were obtained after the previous amplification reaction steps, and a purification operation is performed, such that the amplified DNA chain is adsorbed onto the magnetic bead. Then the liquid is driven to the waste liquid region, leaving the magnetic bead with the amplified DNA chain.

Referring to FIG. 12I, an elution buffer droplet is generated by the droplet generation structure, and the elution buffer droplet is driven to move to the reaction region C of the droplet receiving module to elute the amplified DNA and obtain the amplified DNA chain. For example, this step may use two droplet generation modules B-2 at the right side. The residual liquid in the droplet generation module B-2 at the bottom right corner is removed, an elution solution is added, and two 2 uL elution droplets SD13 are generated by the droplet generation module. These elution droplets SD13 are then driven to move to the reaction region C, followed by magnetic operation where the magnetic bead is discarded and the liquid product (including the amplified DNA chain) is retained. The liquid product is then moved to the droplet generation module B-2 at the top right corner, from which the liquid product is extracted, thereby completing the preparation of the gene library.

It should be understood that FIG. 12A to FIG. 12I are only one example of application of the microfluidic chip, and the present disclosure is not limited thereto. The microfluidic chip of the embodiments of the present disclosure may be applied to any suitable field, and may realize the manipulation of droplets of multiple types and a wide span of volumes.

The following statements should be noted:

(1) The drawings of the present disclosure involve only the structure(s) in connection with the embodiment(s) of the present disclosure, and other structure(s) can be referred to common design(s).

(2) In case of no conflict, features in one embodiment or in different embodiments can be combined to obtain new embodiments.

What have been described above are only specific implementations of the present disclosure, the protection scope of the present disclosure is not limited thereto. Any modifications or substitutions easily occur to those skilled in the art within the technical scope of the present disclosure should be within the protection scope of the present disclosure. Therefore, the protection scope of the present disclosure should be based on the protection scope of the claims.

1. A digital microfluidic chip, comprising:
a droplet generation structure, configured to generate droplets of various volumes, and the droplet generation structure comprises:
one or more droplet generation modules, wherein each droplet generation module is configured to generate at least one of a first droplet, a second droplet, and a third droplet, and the first droplet, the second droplet, and the third droplet respectively have a first volume, a second volume, and a third volume that are different from each other;
a droplet receiving module, configured to achieve at least one of mixing and reaction of a plurality of droplets; and
an interconnection module, configured to drive the first droplet, the second droplet, and the third droplet, and to connect the droplet generation structure to the droplet receiving module.

2. The digital microfluidic chip according to claim 1, wherein the interconnection module comprises:
one or more droplet driving modules, wherein each droplet driving module is configured to drive at least one of the first droplet, the second droplet, and the third droplet, and
a volume of a drivable droplet of a single driving electrode in the one or more droplet driving module is the first volume, the second volume, or the third volume.

3. The digital microfluidic chip according to claim 2, wherein the one or more droplet driving modules comprise:
at least one first droplet driving module, wherein a single driving electrode in each first droplet driving module is configured to drive the first droplet;
at least one second droplet driving module, wherein a single driving electrode in each second droplet driving module is configured to drive the second droplet; and
at least one third droplet driving module, wherein a single driving electrode in each third droplet driving module is configured to drive the third droplet.

4. The digital microfluidic chip according to claim 3, wherein the second volume is larger than a minimum volume of a drivable droplet of the first droplet driving module, and is smaller than a maximum volume of the drivable droplet of the first droplet driving module; and
the third volume is larger than a minimum volume of a drivable droplet of the second droplet driving module, and is smaller than a maximum volume of the drivable droplet of the second droplet driving module.

5. The digital microfluidic chip according to claim 1, wherein the first volume is smaller than the second volume and the third volume, both the second volume and the third volume are integer multiples of the first volume, and the second volume and the third volume are different from each other.

6. (canceled)

7. The digital microfluidic chip according to claim 1, wherein the one or more droplet generation modules comprise:
at least one first droplet generation module, wherein each first droplet generation module is configured to generate the first droplet;
at least one second droplet generation module, wherein each second droplet generation module is configured to generate the second droplet; and
at least one third droplet generation module, wherein each third droplet generation module is configured to generate the third droplet.

8. The digital microfluidic chip according to claim 7,
wherein each of the first droplet generation module, the second droplet generation module, and the third droplet generation module comprises a first driving electrode, a second driving electrode, a third driving electrode, a fourth driving electrode, a fifth driving electrode, and a sixth driving electrode; and
a volume of a droplet that can be accommodated and driven by a driving electrode with a smallest area in each droplet generation module is approximately equal to a volume of a single droplet generated by the each droplet generation module.

9. The digital microfluidic chip according to claim 8, wherein in each of the first droplet generation module, the second droplet generation module, and the third droplet generation module, the second driving electrode, the third driving electrode, the fourth driving electrode, and the sixth driving electrode each have a first area and are arranged sequentially in a first sub-direction, and the sixth driving electrode is connected to a driving electrode in the interconnection module;
the fifth driving electrode comprises two fifth driving sub-electrodes that are electrically connected to each other, and the two fifth sub-electrodes are arranged on opposite sides of the second driving electrode and the third driving electrode in a second sub-direction perpendicular to the first sub-direction, and each of the two fifth driving sub-electrodes has a second area; and
the first driving electrode is disposed on a side of the second driving electrode away from the third driving electrode in the first sub-direction, and is connected to the second driving electrode and the fifth driving electrode, and the first driving electrode has a third area,
wherein the first area is smaller than the second area, and the second area is smaller than the third area.

10. The digital microfluidic chip according to claim 9, wherein each of the first droplet generation module and the second droplet generation module further comprises an additional driving electrode, the additional driving electrode is located on a side of the first driving electrode away from the second driving electrode and the fifth driving electrode, and the additional driving electrode has the third area same as that of a corresponding first driving electrode.

11. The digital microfluidic chip according to claim 10, wherein each of the first droplet generation module and the second droplet generation module further comprises an additional dummy electrode, and the additional dummy electrode is located on a side of the additional driving electrode away from the first driving electrode.

12. (canceled)

13. The digital microfluidic chip according to claim 1, wherein the one or more droplet generation modules comprise:
at least one fourth droplet generation module, wherein each fourth droplet generation module is configured to generate at least two of the first droplet, the second droplet, and the third droplet.

14-17. (canceled)

18. The digital microfluidic chip according to claim 1, wherein the droplet receiving module is configured to at least implement mixing and a first reaction of liquid within a first mixing volume range, and implement mixing and a second reaction of liquid within a second mixing volume range.

19. The digital microfluidic chip according to claim 18, wherein the droplet receiving module comprises:
a first reaction region, configured to at least implement the mixing and the first reaction of the liquid within the first mixing volume range, wherein the first reaction region is connected to a third droplet driving module of the interconnection module.

20. (canceled)

21. The digital microfluidic chip according to claim 19,
wherein the digital microfluidic chip further comprises: a second reaction region, configured to implement the mixing and the second reaction of the liquid within the second mixing volume range, wherein the second reaction region comprises a plurality of second reaction electrodes, and a volume of a droplet that can be accommodated and driven by each second reaction electrode is an integer multiple of the third volume; or
wherein the first reaction region is served as a common reaction region, and is configured to implement the mixing and the first reaction of the liquid within the first mixing volume range, and implement the mixing and the second reaction of the liquid within the second mixing volume range in different operational steps.

22-26. (canceled)

27. The digital microfluidic chip according to claim 2, wherein the droplet generation structure further comprises a droplet cutting module, connected to the one or more droplet generation modules, and is configured to cut a droplet from the one or more droplet generation modules into a droplet having a target volume.

28-36. (canceled)

37. The digital microfluidic chip according to claim 1, comprising:
a first substrate, comprising:
a first base substrate;
a first conductive layer, located on a side of the first base substrate;
a second conductive layer, located on a side of the first conductive layer away from the base substrate, wherein corresponding portions of the first conductive layer and the second conductive layer are electrically connected to each other;
a dummy layer, located between the first conductive layer and the second conductive layer, and being electrically floating or grounded; and
a first hydrophobic layer, located on a side of the second conductive layer away from the first base substrate.

38. The digital microfluidic chip according to claim 37, wherein the second conductive layer comprises a plurality of driving electrodes of the droplet generation structure, the droplet receiving module, and the interconnection module, and the plurality of driving electrodes are spaced apart from each other; and
the dummy layer comprises a plurality of dummy electrodes spaced apart from each other, and an outer contour of an orthogonal projection of each dummy electrode on the first base substrate coincides with an outer contour of an orthographic projection of a corresponding driving electrode among the plurality of driving electrodes on the base substrate.

39-43. (canceled)

44. The digital microfluidic chip according to claim 37, further comprising:
a second substrate, arranged opposite to the first substrate, wherein the second substrate comprises:
a second base substrate;
a third conductive layer, located on a side of the second base substrate; and
a second hydrophobic layer, located on a side of the third conductive layer away from the second base substrate; and
a droplet channel, located between the first hydrophobic layer of the first substrate and the second hydrophobic layer of the second substrate,
wherein the second substrate is provided with a plurality of sample inlets penetrating through the second substrate, and the plurality of sample inlets are respectively arranged corresponding to the plurality of droplet generation modules.

45. (canceled)

46. A method for generating droplets of various volumes using the droplet generation structure in the digital microfluidic chip according to claim 1, comprising at least one of the following:
generating one or more first droplets each having the first volume using the droplet generation module;
generating one or more second droplets each having the second volume using the droplet generation module;
generating one or more third droplets each having the third volume using the droplet generation module, wherein the first volume is smaller than the second volume, and the second volume is smaller than the third volume;
generating at least one of one or more fourth droplets each having a fourth volume and one or more fifth droplets each having a fifth volume using the droplet generation module and a droplet cutting module in combination, wherein the fourth volume and the fifth volume are greater than the second volume and smaller than the third volume; and
mixing at least two of the one or more first droplets, the one or more second droplets, the one or more third droplets, the one or more fourth droplets, and the one or more fifth droplets to obtain droplets with other target volumes.

47-48. (canceled)

49. A method for preparing a gene library using the digital microfluidic chip according to claim 1:
adding a deoxyribonucleotide sample solution, a ligase buffer solution, a linker solution, and a ligase solution to corresponding droplet generation modules of the one or more droplet generation modules, respectively, and generating a sample droplet, a ligase buffer droplet, a linker droplet, and a ligase droplet through the droplet generation structure;
moving the sample droplet, the ligase buffer droplet, the linker droplet, and the ligase droplet to the droplet receiving module through the interconnection module, wherein the sample droplet, the ligase buffer droplet, the linker droplet, and the ligase droplet are mixed and reacted in a first reaction region of the droplet receiving module to form a sample mixture droplet;
performing deoxyribonucleotide adsorption and elution on the sample mixture droplet to obtain a first deoxyribonucleotide chain;
adding an amplification solution and a primer solution to corresponding droplet generation modules among the one or more droplet generation modules, respectively, and generating an amplification droplet and a primer droplet through the droplet generation structure;
moving the amplification droplet and the primer droplet to the droplet receiving module through the interconnection module, wherein the amplification droplet and the primer droplet with the first deoxyribonucleotide chain undergo a polymerase chain reaction in the droplet receiving module to form an amplified deoxyribonucleotide mixture droplet; and
performing deoxyribonucleotide adsorption and elution on the amplified deoxyribonucleotide mixture droplet to obtain an amplified deoxyribonucleotide chain.

50-51. (canceled)
